(19) United States
(12) Patent Application Publication
(10) Pub. No.: US 2025/0144210 A1
(43) Pub. Date: May 8, 2025

(54) SIMPLIFIED METHOD OF PREPARING CELLS FOR PATIENT ADMINISTRATION

(71) Applicant: Seattle Children’s Hospital d/b/a Seattle Children’s Research Institute, Seattle, WA (US)

(72) Inventor: Adam Beebe, Edmonds, WA (US)

(73) Assignee: Seattle Children’s Hospital d/b/a Seattle Children’s Research Institute, Seattle, WA (US)

(21) Appl. No.: 18/835,253

(22) PCT Filed: Feb. 1, 2023
(86) PCT No.: PCT/US2023/061739

§ 371 Date: Aug. 1, 2024

Related U.S. Application Data
(60) Provisional application No. 63/305,358, filed on Feb. 1, 2022.

(51) Int. Cl. A61K 40/11; A61K 40/31; A61K 40/42; C07K 14/725; C07K 16/32; C12N 5/00

(52) U.S. Cl. CPC A61K 40/11; A61K 40/31; A61K 40/4205; C07K 14/7051; C07K 16/32; C12N 5/522; C12N 5/562

(57) ABSTRACT

The current disclosure describes simplified methods of preparing cells for patient infusion where the simplified methods result utilize less steps than conventional methods, decreasing required manipulation steps and reducing the time between beginning of cell manipulation for administration and ultimate administration to a patient. Methods of cryopreserving, thawing, and diluting cells and kits for practicing the methods are also provided herein.

### CROSS-REFERENCE TO RELATED APPLICATION

This application is a U.S. National Phase Application based on International Patent Application No. PCT/US2023/061739, filed on Feb. 1, 2023, which claims priority to U.S. Provisional Patent Application No. 63/305,358 filed Feb. 1, 2022, each of which is incorporated herein by reference in its entirety as if fully set forth herein.

### FIELD OF THE DISCLOSURE

The current disclosure describes simplified methods of preparing cells for patient administration. Methods of modifying, cryopreserving, thawing, and diluting cells and kits for practicing the methods are also provided herein.

### BACKGROUND OF THE DISCLOSURE

Administration of cellular compositions has been effective in the treatment of various pathologies or disorders and has become increasingly prevalent in improving a multitude of therapies. For example, adoptive cell therapy (ACT), involving the administration of lymphocytes (e.g., genetically modified T cells), has shown promise in treating cancer, currently the leading cause of death worldwide. In ACT, a patient's own immune cells can be isolated, genetically-modified, expanded, and reinfused into the patient such that the infused cells elicit a cytotoxic effect on targeted cells (e.g., cancer cells). Because the infused cells can expand, engraft, and persist in vivo, ACT can offer a durable and persistent treatment option for many patients. The process of manufacturing or preparing cells for ACT involves isolating cells from a patient.

The ability to stably store viable cells and to then prepare them and deliver them to patients is important. Additives to the compositions for cryopreservation must be effective to preserve viability and also biologically acceptable when ultimately delivered to the patient.

Many forms of ACT and other cellular therapies cryopreserve cells at a stage during the manufacturing process. Cryopreservation is a process in which cells are preserved by cooling them to low temperatures. At these low temperatures, biological activity, including the biochemical reactions that would lead to cell death under normal conditions, are effectively stopped. Both the cryopreservation process and thaw process are important to ensure proper recovery and cell viability while avoiding cell damage during manufacturing. During the thaw process, cell viability can suffer due to dehydration, toxic solute levels, formation of intracellular ice crystals, and osmotic stress. Current thaw, wash, and preparation for administration processes require several centrifugation steps, a series of sterile welding and sealing steps, taking a count sample in real time, and determining dose based on the processed cell suspension outcome. There is an unmet need for methods that simplify the steps of preparing the cells for administration while maintaining cell viability and sterility.

### SUMMARY OF THE DISCLOSURE

The current disclosure describes simplified methods of preparing cells for administration to a subject. The methods utilize less steps than conventional methods, decreasing required manipulation steps and reducing the time between beginning of cell manipulation for administration and ultimate administration to a patient.

In particular embodiments, the methods include transferring a volume of cells into a first sterile receptacle to create a cell-filled sterile receptacle; transferring a specified volume of diluent into a second sterile receptacle to create a diluent-filled sterile receptacle; connecting the cell-filled sterile receptacle to the diluent-filled sterile receptacle; mixing the volume of cells with the volume of diluent; and transferring the total volume of cells and diluent into either one of the two sterile receptacles to create a product-filled sterile receptacle.

In particular embodiments, the methods include transferring a volume of cells and optionally a volume of diluent into a first sterile receptacle; transferring a specified volume of diluent and optionally a volume of cells into a second sterile receptacle; connecting the first sterile receptacle to the second sterile receptacle; mixing the total volume of cells with the total volume of diluent; and transferring the total volume of cells and diluent into either one of the two sterile receptacles to create a product-filled sterile receptacle.

In particular embodiments, the methods include transferring a volume of cells into a first syringe to create a cell-filled syringe; transferring a specified volume of diluent into a second syringe to create a diluent-filled syringe; connecting the cell-filled syringe to the diluent-filled syringe; mixing the volume of cells with the volume of diluent; and transferring the total volume of cells and diluent into either one of the two syringe to create a product-filled syringe ready for administration to a subject.

In particular embodiments, the methods include attaching a needle to a first syringe, wherein the syringe has a plunger; transferring, through the needle, a specified volume of cells into the first syringe to create a cell-filled syringe; attaching a needle to a second syringe wherein the syringe has a plunger; transferring, through the needle, a specified volume of diluent into the second syringe to create a diluent-filled syringe; removing the needles from both the cell-filled syringe and the diluent-filled syringe; connecting the cell-filled syringe to a first port of a fluid dispensing connector; connecting the diluent-filled syringe to a second port of the fluid dispensing connector; mixing the volume of cells with the volume of diluent by consecutively compressing the plungers of the cell-filled syringe and diluent-filled syringe a plurality of times in any order until the cells and diluent are thoroughly mixed; and transferring the total volume of cells and diluent into either one of the two syringes to create a product-filled syringe.

In particular embodiments, the cells are cryopreserved cells and are thawed prior to use in the methods. In particular embodiments, the thawing includes using an automated thawing device. In particular embodiments, the cells are T cells genetically modified to express a therapeutic molecule. In particular embodiments, the first and second sterile receptacles are syringes that can be fit with a needle. In particular embodiments, the needle is an 18 gauge safety-shielded needle. In particular embodiments, the diluent is an isotonic solution. In particular embodiments, the isotonic solution includes Normosol®-R (ICU Medical, Inc., Clemente, CA). In particular, 100 mL of isotonic solution includes 526 mg sodium chloride, 222 mg sodium acetate, 502 mg sodium gluconate, 37 mg potassium chloride, and 30 mg magnesium chloride hexahydrate. In particular embodiments, the cell-filed sterile receptacle and diluent-filled sterile receptacle are connected with a fluid dispensing connector. In particular embodiments, the specified volume of diluent results in a ratio of cells to diluent of 1:10 (v/v). In particular embodiments, the product-filled sterile receptacle is transferred to a clinical setting for administration to the subject.

Methods of modifying, cryopreserving, thawing, and diluting cells and kits for practicing the methods are also provided herein.

### BRIEF DESCRIPTION OF THE FIGURES

FIG. 1. Flow diagram showing how cells are transferred into a sterile receptacle and diluent is transferred into a different sterile receptacle. The two sterile receptacles are connected and the contents mixed. Finally, the mixed contents are transferred to one of the sterile receptacles.

FIGS. 2A-2G. Figure depicts (2A) syringes piercing the septum of a cell vial and diluent to transfer the contents to the syringe. (2B) The cell-filled syringe is connected to the diluent-filled syringe and (2C) the plungers are compressed to cause mixing of the contents of each syringe into the other syringe. (2C-2E) There is continued mixing of the contents of the two syringes by compressing the plungers to move the contents from one syringe to the other. (2F) All of the contents of both syringes is transferred into one of the two syringes. (2G) The cell and diluent filled syringe is disconnected from the connector and/or other syringe.

FIGS. 3A-3C. Cell Product Viability and Toxicity. Table shows the viability and toxicity of (3A) vials that were infused per Standard Operating Procedures (SOP), (3B) vials approved for infusion, and (3C) vials discarded due to less than 65% viability. † indicates the highest grade experienced between the timepoint infusion and the next infusion. If the timepoint infusion is the subject's last infusion, the highest grade within 7 days post infusion is shown. Blank cells indicate no adverse event of that. * Indicates the adverse event described in this cell had an Unrelated or Unlikely relationship attribution to investigational product.

FIG. 4. Inter-operator Variability. Four individuals each thawed two concentrated CAR T cell products (PD0222 and PD0290) and performed cell counts after making a 1:10 dilution in Normasol. The cell counts per 0.1 mL for each of the four individuals is shown for both cell products including the average and standard deviation.

FIG. 5. Intra-operator Variability. One individual performed multiple 1:10 dilutions of a thawed CAR-T product (PD0270) and the intra-operator precision is shown.

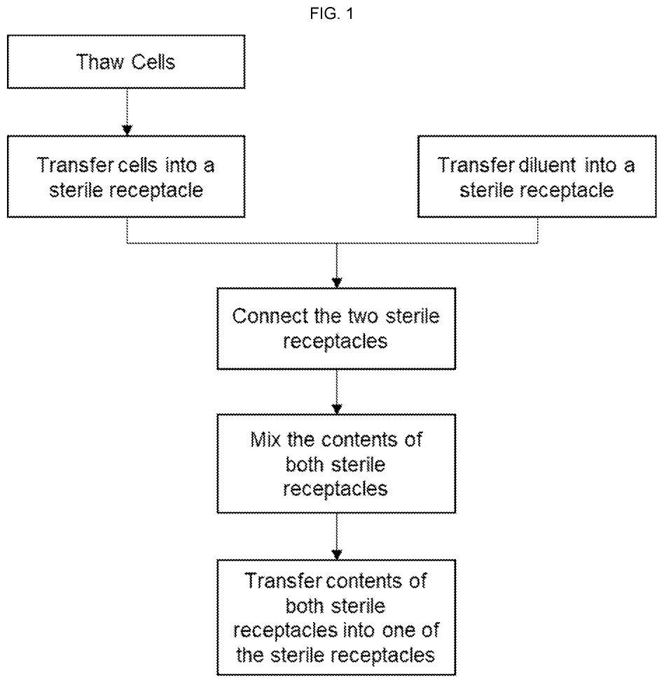

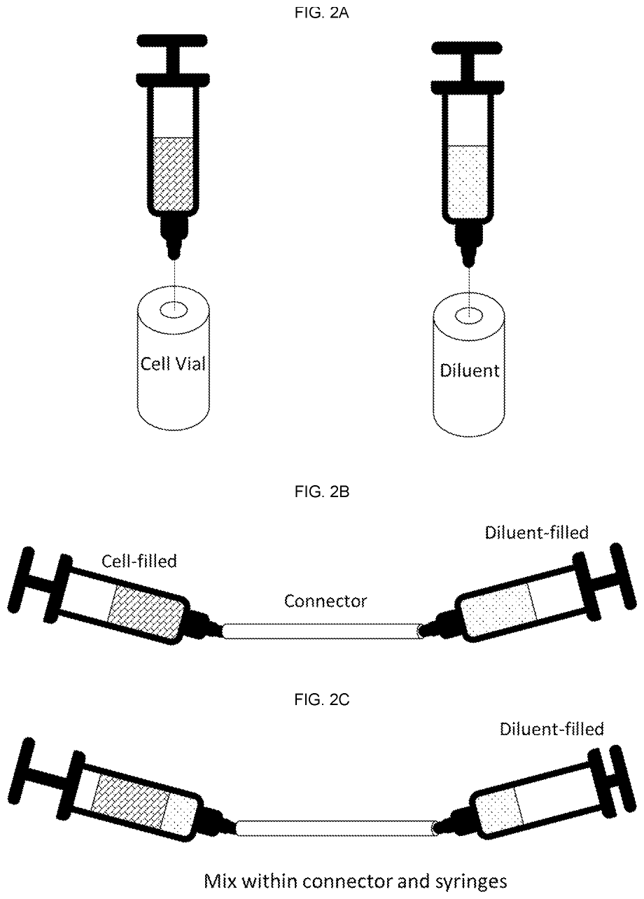

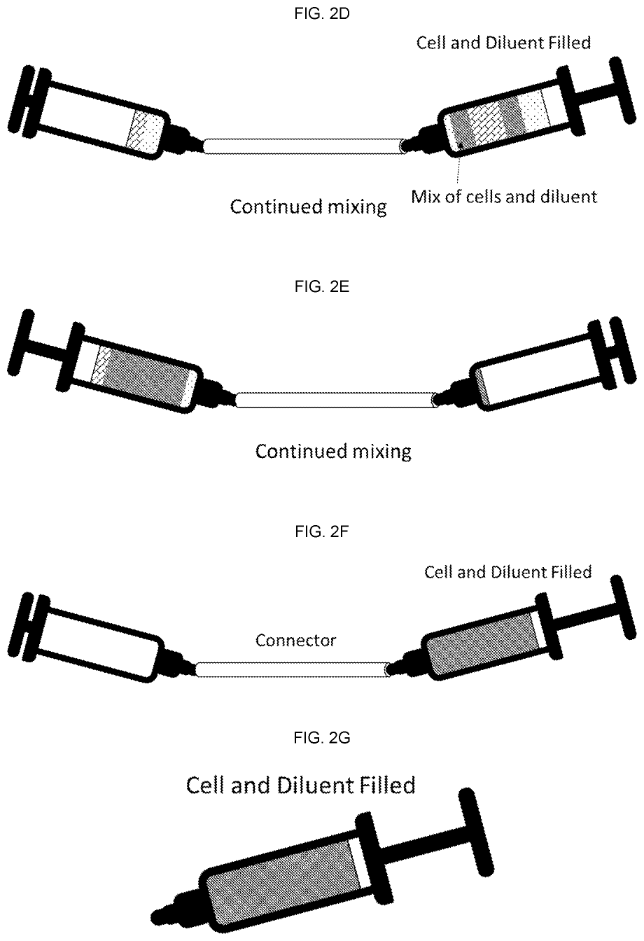

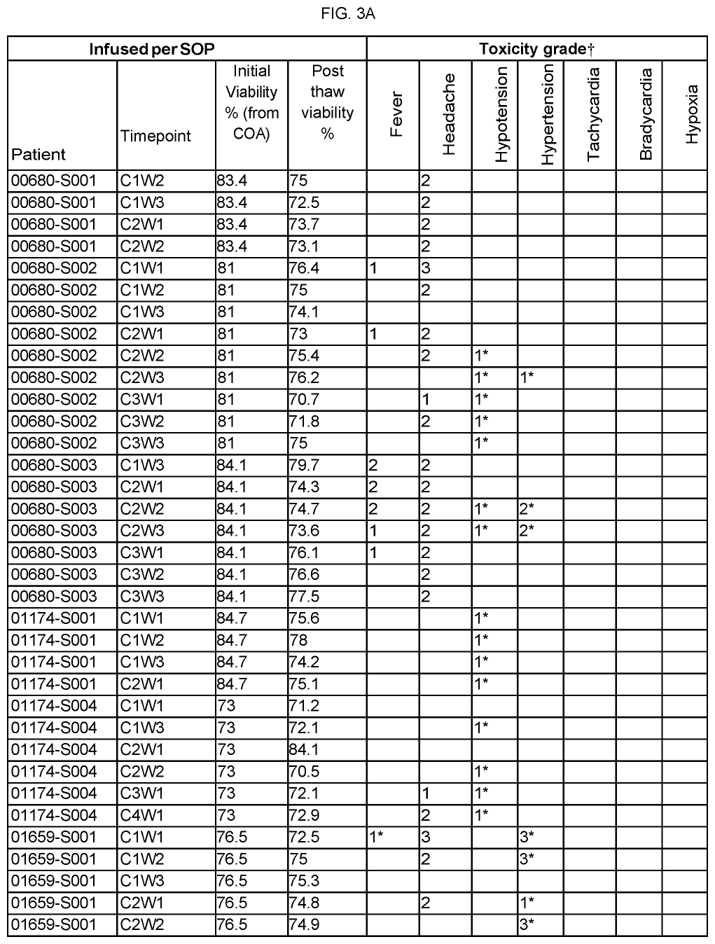

### DETAILED DESCRIPTION

Administration of cellular compositions has been effective in the treatment of various pathologies or disorders and has become increasingly prevalent in improving a multitude of therapies. The ability to stably store viable cells and to then prepare and deliver these cells to patients is important. Cryopreservation compositions must be effective in preserving viability and must also be biologically acceptable when ultimately delivered to the patient.

Cryopreservation is a process in which cells are preserved by cooling them to low temperatures. At these low temperatures, biological activity, including the biochemical reactions that would lead to cell death under normal conditions, are effectively stopped. Both the cryopreservation process and thaw process are important to ensure proper recovery and cell viability while avoiding cell damage. During the thaw process, cell viability can suffer due to dehydration, toxic solute levels, formation of intracellular ice crystals, and osmotic stress. Current thaw and administration preparation processes require several centrifugation steps, a series of sterile welding and sealing steps, taking a sample count in real time, and determining the dose based on the processed cell suspension. Maintaining the cell viability and sterility during preparation of cell formulations for administration is of great concern.

The current disclosure describes simplified methods of preparing cells for administration to a subject. In particular embodiments, the methods include transferring a volume of cells into a first sterile receptacle to create a cell-filled sterile receptacle; transferring a specified volume of diluent into a second sterile receptacle to create a diluent-filled sterile receptacle; connecting the cell-filled sterile receptacle to the diluent-filled sterile receptacle; mixing the volume of cells with the volume of diluent; and transferring the total volume of cells and diluent into either one of the two sterile receptacles to create a product-filled sterile receptacle.

In particular embodiments, the methods include transferring a volume of cells into a first syringe to create a cell-filled syringe; transferring a specified volume of diluent into a second syringe to create a diluent-filled syringe; connecting the cell-filled syringe to the diluent-filled syringe; mixing the volume of cells with the volume of diluent; and transferring the total volume of cells and diluent into either one of the two syringe to create a product-filled syringe ready for administration to a subject.

In particular embodiments, the methods include transferring a volume of cells and optionally a volume of diluent into a first sterile receptacle; transferring a specified volume of diluent and optionally a volume of cells into a second sterile receptacle; connecting the first sterile receptacle to the second sterile receptacle; mixing the total volume of cells with the total volume of diluent; and transferring the total volume of cells and diluent into either one of the two sterile receptacles to create a product-filled sterile receptacle.

In particular embodiments, the methods include attaching a needle to a first syringe, wherein the syringe has a plunger; transferring, through the needle, a specified volume of cells into the first syringe to create a cell-filled syringe; attaching a needle to a second syringe wherein the syringe has a plunger; transferring, through the needle, a specified volume of diluent into the second syringe to create a diluent-filled syringe; removing the needles from both the cell-filled syringe and the diluent-filled syringe; connecting the cell-filled syringe to a first port of a fluid dispensing connector; connecting the diluent-filled syringe to a second port of the fluid dispensing connector; mixing the volume of cells with the volume of diluent by consecutively compressing the plungers of the cell-filled syringe and diluent-filled syringe a plurality of times in any order until the cells and diluent are thoroughly mixed; and transferring the total volume of cells and diluent into either one of the two syringes to create a product-filled syringe.

In particular embodiments, methods for formulation of therapeutic cells for administration to a subject include thawing a vial of cryopreserved cells, disinfecting the entry port of the vial, disinfecting the entry port of the container of diluent, transferring a volume of cells into a first sterile receptacle to create a cell-filled sterile receptacle, transferring a specified volume of diluent into a second sterile receptacle to create a diluent-filled sterile receptacle, connecting the cell-filled sterile receptacle to the diluent-filled sterile receptacle using a fluid dispensing connector, mixing the volume of cells within the volume of diluent within the two sterile receptacles and the fluid dispensing connector, transferring the total contents of cells and diluent into either the first or second sterile receptacle to create a product-filled sterile receptacle.

In particular embodiments, the cells are T cells genetically modified to express a therapeutic molecule. In particular embodiments, the thawing includes using an automated thawing device. In particular embodiments, the cells are cryopreserved at 10×10⁶ cells/ml to 325×10⁶ cells/ml. In particular embodiments, the first and second sterile receptacles are syringes that can be fit with a needle. In particular embodiments, the needle is an 18 gauge safety-shielded needle. In particular embodiments, the diluent is an isotonic solution. In particular embodiments, the isotonic solution includes Normosol®-R (ICU Medical, Inc., Clemente, CA). In particular, 100 mL of isotonic solution includes 526 mg sodium chloride, 222 mg sodium acetate, 502 mg sodium gluconate, 37 mg potassium chloride, and 30 mg magnesium chloride hexahydrate. In particular embodiments, the cell-filed sterile receptacle and diluent-filled sterile receptacle are connected with a fluid dispensing connector. In particular embodiments, the specified volume of diluent results in a ratio of cells to diluent of 1:10 (v/v). In particular embodiments, the product-filled sterile receptacle is transferred to a clinical setting for administration to the subject. In particular embodiments, the product-filled syringe is administered to a subject. In particular embodiments, administration to the subject includes infusion.

In particular embodiments, the therapeutic cells are the cells in the product-filled syringe or product-filled sterile receptacle. In particular embodiments, the therapeutic cells, cryopreserved cells, and/or cell product include immune cells. A cell product refers to cells for therapeutic use including, in certain examples, cells that are genetically modified. In particular embodiments, the immune cells include T cells or genetically modified T cells. In particular embodiments, the genetically modified T cells include T cells genetically modified to express a therapeutic molecule. In particular embodiments, the therapeutic molecule is an anti-Her2 chimeric antigen receptor.

In particular embodiments, the administration includes infusion or injection.

In particular embodiments, the first sterile receptacle and/or second sterile receptacle include a syringe, pipette, dropper, or injector. In particular embodiments, the first sterile receptacle is a syringe. In particular embodiments, the second sterile receptacle is a syringe. In particular embodiments, the first and/or second sterile receptacle are fitted with a needle. In particular embodiments, the needle is a safety-shielded needle. In particular embodiments, the needle is a 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, or a 27 gauge needle. In particular embodiments, the needle is an 18 gauge needle. In particular embodiments, if the sterile receptacle is a syringe, the mixing includes pushing the plunger of the syringe. In other embodiments, mixing includes causing flow between the fluid dispensing connector and sterile receptacles such that the contents become substantially homogenously mixed.

In particular embodiments, the specified volume of diluent is at least 1 times, at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times, or at least 15 times the volume of cells. In particular embodiments, the specified volume of diluent includes 9 times the volume of cells. In particular embodiments, the specified volume of diluent results in a cell to diluent ratio of 1:1 (v/v), 1:2 (v/v), 1:3 (v/v), 1:4 (v/v), 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v). In particular embodiments, the specified volume of diluent results in a cell to diluent ratio of 1:10 (v/v).

In particular embodiments, a fluid dispensing connector includes any means having at least two ports by which fluid is transferred between at least two receptacles connected to the ports in a sterile manner. In particular embodiments, a fluid dispensing connector includes sterile tubing or a sterile fluid lock between sterile receptacles.

In particular embodiments, the diluent includes isotonic solution. In particular embodiments, the isotonic solution includes Normosol®-R (ICU Medical, Inc., Clemente, CA). In particular, 100 mL of isotonic solution includes 0-1000 mg sodium chloride, 0-1000 mg sodium acetate, 0-1000 mg sodium gluconate, 0-1000 mg potassium chloride, and 0-1000 mg magnesium chloride hexahydrate. In particular, 100 mL of isotonic solution includes 250-750 mg sodium chloride, 150-400 mg sodium acetate, 250-750 mg sodium gluconate, 10-80 mg potassium chloride, and 10-50 mg magnesium chloride hexahydrate. In particular, 100 ml of isotonic solution includes 526 mg sodium chloride, 222 mg sodium acetate, 502 mg sodium gluconate, 37 mg potassium chloride, and 30 mg magnesium chloride hexahydrate. In particular embodiments, isotonic solution includes HCl and/or NaOH for pH adjustment.

In particular embodiments, the total volume of cells and diluent is less than 15 ml, less than 14 ml, less than 13 ml, less than 12 ml, less than 11 ml, less than 10 ml, less than 9 ml, less than 8 ml, less than 7 ml, less than 6 ml, less than 5 ml, less than 4 ml, less than 3 ml, less than 2 ml, or less than 1 ml. In particular embodiments, the total volume of cells and diluent is less than 5 ml.

In particular embodiments, disinfecting includes swabbing, spraying, or exposing with or to disinfectant. In particular embodiments, disinfectant includes povidone-iodine, isopropyl alcohol, ethanol, methanol, and/or hydrogen peroxide.

In particular embodiments, thawing a vial of cryopreserved cells includes using an automated thawing device. In particular embodiments, the product-filled sterile receptacle is plugged and transferred to a clinical study for administration to the subject. In particular embodiments, the subject is a human in need of treatment. In particular embodiments, the subject has cancer. In particular embodiments, the cancer includes a central nervous system tumor, a glioma, an ependymoma, a medulloblastoma, a germ cell tumor, an atypical teratoid, a primitive neuroectodermal tumor, a choroid plexus carcinoma, or a pineoblastoma. In particular embodiments, the subject is between 1 and 26 years old.

In particular embodiments, methods for formulation of therapeutic cells for administration to a subject include thawing a vial of cryopreserved cells using an automated thawing device to create a vial of cell product; once thawed, disinfecting the entry port of the vial by swabbing it with povidone-iodine and isopropyl alcohol swabs and allowing the entry port to air dry; disinfecting the entry port of the container of diluent by swabbing it with povidone-iodine and isopropyl alcohol swabs and allowing the entry port to air dry; fitting a first syringe with a needle; piercing the entry port of the vial of cell product with the needle; withdrawing the specified volume of cell product in the first syringe to create a cell-filled syringe; fitting a second syringe with a needle; piercing the entry port of the container of diluent; withdrawing the specified volume of diluent necessary to perform a 1:10 dilution of the cell product with diluent to create a diluent-filled syringe; replacing the needle on the cell-filled syringe with a fluid dispensing connector having a first and second port; removing the needle on the diluent-filled syringe and connecting the diluent-filled syringe to the second port of the fluid dispensing connector; mixing the cell product and diluent between the syringes and fluid dispensing connector; transferring all of the volume of cell product and diluent into one of the two connected syringes creating a product syringe; removing the fluid dispensing connector from the product syringe; and placing a luer plug on the product syringe. In particular embodiments, the product syringe is transferred to a clinical setting for administration to a subject.

In particular embodiments, kits for practicing the methods include at least two sterile fluid receptacles with plungers, at least two needles, and a fluid dispensing connector. In particular embodiments, the kits further include diluent. In particular embodiments, the diluent is an isotonic solution. In particular embodiments, the isotonic solution is Normosol®-R (ICU Medical, Inc., Clemente, CA). In particular embodiments, the needles include 14-27 gauge, e.g. 18 gauge, safety-shielded needles. In particular embodiments, the kits include a luer plug. In particular embodiments, the kits include disinfectant. In particular embodiments, the kits include instructions for performing the methods described herein.

This simplified methods of preparing cells into formulations for administration to a subject overcomes the complications of previous methods which require many centrifugation steps, a series of sterile welding and sealing steps, and taking sample counts in real time. The process described herein reduces the impact on the cells post thaw and allows use of quality control release assay data to determine volume and viability, thus reducing the real time needs during the thaw process. Furthermore, the simplified methods maintain sterility and viability while simplifying and reducing the time needed to prepare cells for administration to a subject.

In certain examples, the scope of the methods described herein focus on the preparation of the formulations for administration and end before the administration to the subject. Although the preparation of the formulations includes the dilution and mixing of the cell product and diluent within sterile receptacles, methods for cryopreserving cells, genetically modifying cells, and thawing cells can affect the final formulations and are therefore also described.

Aspects of the current disclosure are now described with additional detail and options as follows: (i) Cell Types; (ii) Harvesting Cells; (iii) Genetically Modifying Cells; (iv) Cryopreservation; (v) Thawing Cryopreserved Cells; (vi) Preparing Formulations for Administration; (vii) Methods of Use; (viii) Kits; (ix) Exemplary Embodiments; (x) Experimental Examples; and (xi) Closing Paragraphs. These headings are provided for organizational purposes only and do not limit the scope or interpretation of the disclosure.

(i) Cell Types. Cells can include any cell isolated from a subject. In particular embodiments, cells are eukaryotic cells. In particular embodiments, cells are mammalian cells. In particular embodiments, cells are human cells. The cells may be selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotropes, lactotrophs, pneumocytes, type I pneumocytes, type II pneumocytes, Clara cells; goblet cells, alveolar macrophages, myocardiocytes, pericytes, gastric cells, gastric chief cells, parietal cells, paneth cells, G cells, D cells, ECL cells, I cells, K cells, S cells, enteroendocrine cells, enterochromaffin cells, APUD cells, liver cells, hepatocytes, Kupffer cells, bone cells, osteoblasts, osteocytes, osteoclasts, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocytes, fibroblasts, tendon cells, podocytes, juxtaglomerular cells, intraglomerular mesangial cells, extraglomerular mesangial cells, kidney cells, macula densa cells, spermatozoa, sertoli cells, leydig cells, oocytes, stem cells, and mixtures thereof. In particular embodiments, stem cells include embryonic stem cells, mesenchymal stem cells, induced pluripotent stem cells, hematopoietic stem cells, neural stem cells, epithelial stem cells, and hematopoietic progenitor cells. Cells lines of any of the cells disclosed herein may also be used with the methods disclosed herein.

In particular embodiments, cells used with the methods disclosed herein include immune cells. Immune cells include T-cells, B cells, natural killer (NK) cells, NK-T cells, monocytes/macrophages, lymphocytes (e.g., tumor-infiltrating lymphocytes (TIL) or marrow-infiltrating lymphocytes (MIL)), hematopoietic stem cells (HSCs), hematopoietic progenitor cells (HPC), induced pluripotent stem cells (iPSC), mucosal-associated invariant T (MAIT) cells, dendritic cells, and/or a mixture of HSC and HPC (i.e., HSPC). In particular embodiments, cells include T cells. In particular embodiments, cells are CD4+ T cells. In particular embodiments, cells are CD8+ T cells. In particular embodiments, cells are CD4+ and CD8+ T cells.

(ii) Harvesting Cells. Cells may be autologous/autogeneic (“self”) or non-autologous (“non-self,” e.g., allogeneic, syngeneic, or xenogeneic) in reference to a particular patient. In particular embodiments, cells are autologous.

Cells may be cells isolated from any tissue or organ (e.g., blood or connective tissue).

Immune cells (e.g., T cells) can be obtained from a number of sources including peripheral blood, peripheral blood mononuclear cells (PBMCs), bone marrow, lymph nodes tissue, cord blood, thymus issue, tissue from a site of infection, ascites, pleural effusion, spleen tissue, and tumors. Immune cells can be obtained from a cell line, for example, T cells may also be obtained from a cultured T cell line (e.g., Jurkat). In particular embodiments, cells are isolated from a sample such as blood or a blood-derived sample.

Blood or blood-derived samples include an apheresis or a leukapheresis product. Exemplary samples include whole blood, peripheral blood mononuclear cells (PBMCs), bone marrow, thymus, cancer tissue, lymphoid tissue, spleen, or other appropriate sources. In particular embodiments, cells are isolated from PBMCs.

In particular embodiments, the volume of apheresed or leukapheresed cells is 5 mL to 1000 mL, 50 mL to 500 mL, 50 mL to 250 mL, 50 mL to 200 mL, 100 mL to 500 mL, 125 mL to 350 mL, 100 mL to 250 mL, or 100 mL to 200 mL, or any intervening range thereof. In particular embodiments, the volume of apheresed cells is 25 mL, 50 mL, 75 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 800 mL, or 1000 mL or any intervening volume thereof.

Methods regarding collection, anti-coagulation and processing, etc. of blood samples can be found in, for example, Alsever, et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160.

In particular embodiments, collected cells are washed, centrifuged, and/or incubated in the presence of one or more reagents, for example, to remove unwanted components, enrich for desired components, lyse or remove cells sensitive to particular reagents. The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker−) or express relatively low levels (marker^{lo}) of one or more markers.

In particular embodiments, a population of cells is isolated and/or purified from another population of cells. In particular embodiments, T cells can be isolated from PBMCs by lysing the red blood cells and depleting the monocytes, for example, by centrifugation through a PERCOLL® (Cytiva Sweden AB, Upsala, Sweden) gradient. A specific subpopulation of T cells, expressing CD4, CD8, CD3, CD28, CD45RA, and/or CD45RO is further isolated by positive or negative selection techniques. In particular embodiments, a specific population of T cells expressing CD4 and CD8 are isolated and selected. In particular embodiments, cell sorting and/or selection occurs via negative magnetic immunoadherence or flow cytometry using a cocktail of monoclonal antibodies directed to cell surface markers present on the cells negatively selected. For example, to enrich for CD4+ cells by negative selection, a monoclonal antibody cocktail that typically includes antibodies to CD14, CD20, CD11b, CD16, HLA-DR, and CD8 can be used.

Following isolation and/or enrichment, cells can be expanded to increase the number of cells. In particular embodiments, cells can be expanded before or after genetic modification. In particular embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, T cells are expanded by contact with a surface having attached thereto an agent that stimulates a CD3 TCR complex associated signal and a ligand that stimulates a co-stimulatory molecule on the surface of the T cells. In particular embodiments, PBMCs or isolated T cells are contacted with a stimulatory agent and costimulatory agent, such as anti-CD3 and/or anti-CD28 antibodies, generally attached to a bead or other surface, in a culture medium with appropriate cytokines (see Berg et al., Transplant Proc. 30 (8): 3975-3977, 1998; Haanen et al., J. Exp. Med. 190 (9): 13191328, 1999; Garland et al., J. Immunol Meth. 227 (1-2): 53-63, 1999). In particular embodiments, the T cells may be activated and stimulated to proliferate with feeder cells and appropriate antibodies and cytokines using methods such as those described in U.S. Pat. Nos. 6,040,177; 5,827,642; and WO 2012/129514.

In particular embodiments, HSPCs can be isolated and/or expanded following methods described in, for example, U.S. Pat. Nos. 7,399,633; 5,004,681; US 2010/0183564; WO2006/047569; WO2007/095594; WO 2011/127470; and WO 2011/127472; Vamum-Finney, et al., 1993, Blood 101:1784-1789; Delaney, et al., 2005, Blood 106:2693-2699; Ohishi, et al., 2002, J. Clin. Invest. 110:1165-1174; Delaney, et al., 2010, Nature Med. 16 (2): 232-236; and Chapter 2 of Regenerative Medicine, Department of Health and Human Services, August 2006, and the references cited therein. The collection and processing of other cell types described herein are known by one of ordinary skill in the art.

In particular embodiments, the isolating, incubating, expansion, and/or any other steps during cell processing are carried out in a sterile or contained environment and/or in an automated fashion, such as controlled by a computer attached to a device in which the steps are performed. Final formulation of cells into formulations for administration is described elsewhere herein.

In particular embodiments, cells can be washed after isolation. In particular embodiments, cells can be counted. In particular embodiments, cell viability can be assessed.

As used herein the term “viability” or “viable” refers to a cell that is capable of normal growth and development. Assessing the viability of a population of cells can be conducted in order to know how many live cells may be administered to a patient. Furthermore, assessment of the viability of cells can be used to compare cryopreserving, processing, thawing, and dilution procedures and their impact on viability.

Examples of experiments that can be used to determine the level of cell viability include trypan blue staining and MTS assays. The MTS assay is a measure of functional viability (i.e. metabolism), while the trypan blue assay measure structural viability (i.e. membrane integrity). Other methods known to those skilled in the art, such as alamar blue assays, may also be used for cell viability measurements.

In particular embodiments, harvested cells can be genetically modified. In particular embodiments, harvested cells can be cryopreserved for future use and/or analysis.

(iii) Genetically Modifying Cells. A cell product refers to a population of cells for therapeutic use. Cell products can include in vitro expanded cell populations and/or engineered cell populations for use in immune therapy, among other uses. In particular embodiments, a cell product includes genetically modified cells.

In particular embodiments, harvested cells may be genetically modified ex vivo. In particular embodiments, harvested cells may be genetically modified to include a desired gene. In particular embodiments, a desired gene can express a therapeutic molecule. In particular embodiments, a therapeutic molecule includes a recombinant molecule that activates a cell upon ligand binding.

Desired genes (e.g. genes encoding a therapeutic molecule) can be introduced into cells by any methods known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, nanoparticle-mediated delivery, mammalian artificial chromosomes (Vos, 1998, Curr. Op. Genet. Dev. 8:351-359), liposomes (Tarahovsky and Ivanitsky, 1998, Biochemistry (Mosc) 63:607-618), ribozymes (Branch and Klotman, 1998, Exp. Nephrol. 6:78-83), triplex DNA (Chan and Glazer, 1997, J. Mol. Med. 75:267-282), etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the desired gene to the cell, so that the desired gene is expressed by the cell and, in certain instances, preferably heritable and expressed in its cell progeny.

In particular embodiments, a desired gene can be introduced into cells in a vector. A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

Viral vectors can be derived from numerous viruses. “Lentivirus” refers to a genus of retroviruses that are capable of infecting dividing and non-dividing cells and typically produce high viral titers. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Additional examples of viral vectors include those derived from foamy viruses, adenoviruses (e.g., adenovirus 5 (Ad5), adenovirus 35 (Ad35), adenovirus 11 (Ad11), adenovirus 26 (Ad26), adenovirus 48 (Ad48) or adenovirus 50 (Ad50)), adeno-associated virus (AAV; see, e.g., U.S. Pat. No. 5,604,090; Kay et al., 2000; Nakai et al., z1998), alphaviruses, cytomegaloviruses (CMV), flaviviruses, herpes viruses (e.g., herpes simplex), influenza viruses, papilloma viruses (e.g., human and bovine papilloma virus; see, e.g., U.S. Pat. No. 5,719,054), poxviruses, vaccinia viruses, etc. See Kozarsky and Wilson, 1993; Rosenfeld, et al., 1991; Rosenfeld, et al., 1992; Mastrangeli, et al., 1993; Walsh, et al., 1993; and Lundstrom, 1999. Examples include modified vaccinia Ankara (MVA) and NYVAC, or strains derived therefrom. Other examples include avipox vectors, such as a fowlpox vectors (e.g., FP9) or canarypox vectors (e.g., ALVAC and strains derived therefrom). For additional information regarding viral vectors for gene delivery, see Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686; Miller, et al., 1993, Meth. Enzymol. 217:581-599); Naldini et al. (1996) Science 272 (5259): 263-267; Naldini et al. (1996) Proceedings of the National Academy of Sciences 93 (21): 11382-11388; Zufferey et al. (1997) Nature biotechnology 15 (9): 871-875; Dull et al. (1998) Journal of virology 72 (11): 8463-8471; U.S. Pat. Nos. 6,013,516; and 5,994,136).

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9 (8): 805-7; Ramirez et al., Nucl Acids Res, 2012, 40 (12): 5560-8; Kim et al., Genome Res, 2012, 22 (7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14 (l): 49-55; Beurdeley et al., Nat Commun, 2013, 4:1762; Scharenberg et al., Curr Gene Ther, 2013, 13 (4): 291-303; Gaj et al., Nat Methods, 2012, 9 (8): 805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

In particular embodiments, a therapeutic molecule includes a chimeric antigen receptor (CAR) or a T cell receptor (TCR). As used herein, a CAR is a synthetically designed protein including a ligand binding domain that binds to an antigen associated with a disease or disorder. The ligand binding domain is linked to one or more intracellular signaling domains of an immune cell. CAR and TCR can include a ligand binding domain, transmembrane domain, and intracellular signaling domain. A CAR or TCR can include linkers, spacers, junction amino acids, tags, and/or selectable markers.

A ligand binding domain is any molecule capable of specifically binding a target antigen. Exemplary ligand binding domains include antibody binding fragments (e.g., scFv), antibodies, receptors (e.g., T cell receptors), and receptor ligands (e.g., a cytokine or chemokine). A ligand binding domain can bind a cancer antigen, a viral antigen, or a B-cell ligand. In particular embodiments, a ligand binding domain binds a cancer antigen. Exemplary cancer antigens include carcinoembryonic antigen (CEA), prostate specific antigen, Prostate Stem Cell antigen (PSCA), PSMA, Her2/neu, estrogen receptor, progesterone receptor, ephrinB2, CD19, CD20, CD22, CD23, CD123, CE7, ROR1, mesothelin, c-Met, GD-2, MAGE A3 TCR, EGFR, EGFRvIII, EphA2, L1CAM, oaGD2, GD2, CD33, FITC, VAR2CSA, PD-L1, ERBB2, folate receptor (FOLR), glypican-2, disialoganglioside, EpCam, L1-CAM, WT-1, Tyrosinase related protein 1 (TYRP1/gp75), B-cell maturation antigen (BCMA), CD24, SV40 T, and CD133. Other examples are known to those of ordinary skill in the art. In particular embodiments, the ligand binding domain specifically binds Her2.

The intracellular signaling domain can include one or more intracellular signaling components. In particular embodiments, the intracellular signaling domain generates a signal that promotes an immune effector function of a therapeutic molecule modified cell. In particular embodiments, the intracellular signaling domain generates a stimulatory and/or co-stimulatory signal based on ligand binding. Examples of immune effector function include cytolytic activity and helper activity, including the secretion of cytokines. Intracellular signaling domain signals can also lead to immune cell proliferation, activation, differentiation, and the like. A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), HVEM (LIGHTR), ICAM-1, lymphocyte function-associated antigen-1 (LFA-1; CD11a/CD18), CD2, CD7, CD287, NKG2C, NKG2D, SLAMF7, NKp30, NKp44, CD160 (BY55), CD19, CD4, CD8α, IL2RB, IL2Rγ, ITGA4, VLA1, IA4, CD49d, VLA-6, CD49f, CD11d, ITGAE, ITGAL, ITGAM, ITGAX, CD11c, CD29, ITGB2, ITGB7, TNFR2, DNAM1 (CD226), SLAMF4 (CD244, 2B4), CD96 (Tactile), CEACAM1, Ly9 (CD229), PSGL1, CD100 (SEMA4D), SLAMF6 (NTB-A, Ly108), SLAM (SLAMF1, CD150, IPO-3), SELPLG (CD162), LTBR, GADS, SLP-76, CD19a, a ligand that specifically binds with CD83, and the like.

Therapeutic molecules can be designed to include a transmembrane domain that links an extracellular component of the therapeutic molecule to an intracellular component of the therapeutic molecule when expressed. A transmembrane domain can anchor a therapeutic molecule to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that the signaling domain, costimulatory domain, or hinge domain is derived from. In particular embodiments, the transmembrane domain is not derived from the same protein that any other domain of a fusion protein is derived from. In particular embodiments, the transmembrane domain can be selected or modified by amino acid substitution to avoid binding of or to minimize interactions with other domains in the fusion protein.

The transmembrane domain may be derived either from a natural or from a recombinant source. Where the source is natural, the domain may be derived from any membrane-bound or transmembrane protein. In particular embodiments, the transmembrane domain is capable of signaling to the intracellular domain(s) whenever a fusion protein having an extracellular ligand binding domain has bound to a target. In particular embodiments, a transmembrane domain may include at least the transmembrane region(s) of: the α, β, or ζ chain of the T-cell receptor; CD28; CD27; CD3ε; CD4; CD5; CD9; CD16; CD33; CD37; CD80; CD86; CD137; and/or CD154. In particular embodiments, a transmembrane domain may include at least the transmembrane region(s) of: KIRDS2; OX40; LFA-1; ICOS; 4-1BB; CD40; BAFFR; SLAMF7; NKp80; NKp30; NKp46; CD19; IL2Rβ; IL7Ra; ITGA1; CD49a; ITGA4; CD49D; VLA-6; CD49f; CDI Id; ITGAE; ITGAL; CDI Ia; CDI Ib; ITGAX; ITGB1; CD29; CD18; ITGB7; DNAM1; SLAMF4; CD96; CRT AM; Ly9; CD160; CD100; SLAMF6 (NTB-A, Lyl08); BLAME; SELPLG; PAG/Cbp; and/or NKG2C.

In particular embodiments, the cell product includes autologous CD4+ and CD8+ T cells genetically modified to express a therapeutic molecule. In particular embodiments, the cell product includes autologous CD4+ and CD8+ T cells genetically modified to express an anti-Her2 therapeutic molecule and EGFRt. In particular embodiments, the cell product includes cells genetically modified to express an anti-Her2 CAR and EGFRt.

(iv) Cryopreservation. During cell processing (e.g. after harvesting, before or after expanding, or before or after genetic modification), the cells can be cryopreserved. The term “cryopreservation” is used to describe the storage of cells in low temperature environments, i.e. −70° C. to −196° C. These temperatures are suitable for long term storage (months to years). The use of the terms “freezing”, to “freeze” and “frozen” in the context of cells as discussed herein refers to the act of exposing the cells to, and cells that have been subjected to, such low temperatures. In particular embodiments, cells are cryopreserved after harvesting the cells. In particular embodiments, the cells are cryopreserved after genetic modification. In particular embodiments, the cells are cryopreserved after expanding.

Freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroy the cell. (For a discussion, see Mazur, 1977, Cryobiology 14:251-272.) These injurious effects can be circumvented by (a) use of a cryopreservative, (b) control of the freezing rate, and (c) storage at a temperature sufficiently low to minimize degradative reactions.

A cryopreserving step can begin by first preparing a freezing medium containing serum and cryopreservative. Aliquots of this freeze mix are prepared under sterile conditions and then stored at −20° C. for use as a suspension medium for freezing cells. Cells suspended in medium are centrifuged. The supernatant is gently discarded, cells are washed and the cell pellet is then suspended in the thawed freeze mix at a specific cell density. Cells can be stored in any sterile container known by those skilled in the art including tubes or vials. In particular embodiments, cells are cryopreserved in a CellSeal® (Sexton Biotechnologies, Inc.) vial.

In certain examples, cryopreservation begins with resuspending cells in a suitable freezing medium. A suitable freezing medium includes medium that is not toxic to cells at room temperature including a physiologically acceptable buffer, e.g. PBS, DMEM, Iscove's etc., glucose at a concentration of from 1000 to 5000 mg/L; and a cryopreservative. The freezing medium may further include at least 10%, at least 20%, not more than 90%, and not more than 25% serum or serum substitute, e.g. fetal bovine serum, albumin, serum replacement, etc.

Exemplary cryopreservatives include dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, or inorganic salts. In particular embodiments, the cryopreservative is a commercially available cryopreservation media. In particular embodiments, the commercially available cryopreservation media is CryoStor® CS5 Freeze Media (BioLife Solutions, Inc., Bothell, WA 98021). CryoStor® CS5 Freeze Media is pre-formulated to include 5% DMSO. In particular embodiments, the cryopreservative is DMSO. Being a small molecule, DMSO freely permeates the cell and protects intracellular organelles by combining with water to modify its freezability and prevent damage from ice formation. Addition of plasma, fetal calf serum, or human albumin can augment the protective effect of DMSO. After addition of DMSO, cells should be kept at 0° C. until freezing, since DMSO concentrations of 1% are toxic at temperatures above 4° C. In particular embodiments, the cryopreservative is present in the freezing medium at 1% to 20% (volume per volume, v/v), or 5% to 15% (v/v). In particular embodiments, the cryopreservative is present in the freezing medium at 1%, 2%, 5%, 10%, 15%, or 20% (v/v). In particular embodiments, the cryopreservative is present in the freezing medium at 10% (v/v).

Suspensions of cells can be aliquoted into a suitable closed container. In particular embodiments, a suitable closed container includes small vials, closed straws, CellSeal® (Sexton Biotechnologies, Inc. Indianapolis, IN) vials, etc.

A controlled slow cooling rate can be critical in cryopreservation. Different cryopreservatives (Rapatz, G., et al., 1968, Cryobiology 5 (1): 18-25) and different cell types have different optimal cooling rates (see, e.g., Rowe and Rinfret, 1962, Blood 20:636; Rowe, 1966, Cryobiology 3:12-18; Lewis et al., 1967, Transfusion 7:17-32; and Mazur, 1970, Science 168:939-949). The heat of fusion phase where water turns to ice should be minimal. The cooling procedure can be carried out by use of, e.g., a programmable freezing device or a methanol bath procedure. Any methods for cryopreserving that preserves viability of the cells can be used. In particular embodiments, a controlled rate freezer is used in the cryopreserving step to bring the temperature of the vial of cells to less than or equal to −80° C., at a rate ranging from −0.3 to −2° C. per minute. By way of example, the following program can be used: 1) wait for chamber to be 4° C. and sample is 6.0° C.; 2) ramp at 1.0° C./min until sample is −6.0° C.; 3) ramp at 25° C./min until chamber is −45° C.; 4) ramp at 10° C./min until chamber is −14° C.; 5) ramp at 1.0° C./min until chamber is −40° C.; 6) ramp at 10° C./min until chamber is −80° C.; and 7) transfer to liquid nitrogen. Alternatively, the cells can be placed in a Mr. Frosty™ (Nalge Nunc International, Rochester, NY) or other alcohol/polystyrene insulated freezing chamber pre-conditioned at −20° C. and frozen overnight by transferring the chamber to a −80° C. freezer, prior to transfer to liquid nitrogen storage.

After thorough freezing, the ex vivo population of cells can be rapidly transferred to a long-term cryogenic storage vessel. The long-term cryogenic storage vessel can include storage in liquid nitrogen (−196° C.) or its vapor (−165° C.). The long-term cryogenic storage vessel can include a freezer at −80° C. for 2 days and then storage in liquid nitrogen. In particular embodiments, cryopreserved cells are stored in a vapor phase liquid nitrogen dewar.

The cell density will be cell dependent. In particular embodiments, the cell density is 1×10⁶ cells/ml, 2×10⁶ cells/ml, 3×10⁶ cells/ml, 4×10⁶ cells/ml, 5×10⁶ cells/ml, 6×10⁶ cells/ml, 7×10⁶ cells/ml, 8×10⁶ cells/ml, 9×10⁶ cells/ml, 10×10⁶ cells/ml, 15×10⁶ cells/ml, 20×10⁶ cells/ml, 30×10⁶ cells/ml, 50×10⁶ cells/ml, 100×10⁶ cells/ml, 150×10⁶ cells/ml, 200×10⁶ cells/ml, 250×10⁶ cells/ml, 300×10⁶ cells/ml, or 350×10⁶ cells/ml. In particular embodiments, the cell density is 10-325 cells/ml.

Further considerations and procedures for the manipulation, cryopreservation, and long-term storage of cells, can be found in the following exemplary references: U.S. Pat. Nos. 4,199,022; 3,753,357; and 4,559,298; Gorin, 1986, Clinics In Haematology 15 (1): 19-48; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987, Nature 327:255; Linner et al., 1986, J. Histochem. Cytochem. 34 (9): 1123-1135; Simione, 1992, J. Parenter. Sci. Technol. 46 (6): 226-32).

(v) Thawing Cryopreserved Cells. If cells are cryopreserved, the cryopreserved cells can undergo thawing steps before they are used for therapeutic administration. If the cryopreservative is toxic in humans, it can be removed prior to therapeutic administration (e.g. during the thawing process). In particular embodiments, the simplified methods of preparing cells for administration do not require cryopreservative to be removed.

Methods for thawing cryopreserved cells are well known in the art (See, e.g., Freshney R I, Culture of Animal Cells: A Manual of Basic Technique, 4^{th} Edition, 2000, Wiley-Liss, Inc., Chapter 19).

It will be appreciated that the thawing rate of cryopreserved cells will be influenced by a variety of factors. For example, the volume of the cryopreserved cells, handling time, ambient temperature, temperature of incubation chambers used, and heat transfer properties of the container housing the cells may influence thawing rate. It will also be appreciated that cells in a particular sample of cryopreserved cells may not all thaw at the same rate or within the same time period.

The cryopreserved cells to be thawed may be in a composition that occupies a volume of up to 0.1 ml, 0.2 ml, 0.3 ml, 0.4 ml, 0.5 ml, 0.6 ml, 0.7 ml, 0.8 ml, 0.9 ml, 1 ml, 1.3 ml, 1.5 ml, 1.7 ml, 2 ml, 2.5 ml, 3 ml, 3.5 ml, 4 ml, 5 ml, 10 ml, 20 ml, 30 ml, 40 ml, 50 ml, or more. The cryopreserved cells may be in a composition that occupies a volume ranging from 0.1 ml to 5 ml, from 5 ml to 20 ml, from 20 ml to 30 ml, from 30 ml to 40 ml, or from 40 ml to 50 ml. The composition including the cells may be a tissue, e.g., a blood sample. The composition including the cells may further include other agents, e.g., cryopreservatives such as DMSO, glycerol, sucrose, or Trehalose. In particular embodiments, cryopreserved cells are in a composition that occupies a volume less than 5 ml. In particular embodiments, cryopreserved cells are in a composition that occupies a volume less than 4 ml. In particular embodiments, cryopreserved cells are in a composition that occupies a volume less than 3 ml. In particular embodiments, cryopreserved cells are in a composition that occupies a volume less than 2 ml. In particular embodiments, cryopreserved cells are in a composition that occupies a volume less than 1 ml.

Typically, the step of thawing involves obtaining cryopreserved cells from storage at a temperature of less than 0° C. (a subzero temperature) and allowing them to come to a temperature above 0° C. The step of thawing may involve obtaining the cryopreserved cells from storage at a temperature that ranges from −205° C. to −195° C. The step of thawing may involve obtaining the cryopreserved cells from storage at a temperature that ranges from −80° C. to −60° C. The step of thawing may involve progressively warming the cryopreserved cells by transferring the cells among incubators each have a warmer temperature range, e.g., to control the rate of thawing. For example, the step of thawing may involve first obtaining cryopreserved cells from storage at a first subzero temperature, e.g., that ranges from −205° C. to −195° C., and transferring the cryopreserved cells to a second, typically warmer, yet typically subzero, storage temperature, e.g., to a temperature that ranges from −80° C. to −60° C., prior to thawing. Any number of stages, e.g., 2, 3, 4, 5, 6, or more stages, are envisioned to control the rate of thawing in this manner. The step of thawing may also involve progressively warming the cryopreserved cells by incubating the cells in a temperature controlled chamber, e.g., a water bath, heat block, oven, etc., and progressively warming the chamber, e.g., at a controlled rate, while the cryopreserved cells are present in the chamber.

The step of thawing may involve incubating the cryopreserved cells at a temperature that ranges from 15° C. to 30° C. The step of thawing may involve incubating the cryopreserved cells at a temperature that ranges from 30° C. to 45° C. In particular embodiments, the step of thawing may involve incubating the cryopreserved cells at a temperature of 37° C. Such incubation may be performed by incubating a container housing the cryopreserved cells in a temperature controlled incubator, e.g., a temperature controlled water bath, a temperature controlled oven, etc. In particular embodiments, the container housing the cryopreserved cells is a vial. A vial is any suitable, sterile vessel for storing cells or cell product. In particular embodiments, the vial is a CellSeal® (Sexton Biotechnologies, Inc.) vial. Other incubation methods will be apparent to the skilled artisan. In particular embodiments, the cryopreserved cells are thawed using an automated thawing system. In particular embodiments, the cryopreserved cells are thawed using a CellSeal® (Sexton Biotechnologies, Inc.) Automated Thawing System. In particular embodiments, the CellSeal® (Sexton Biotechnologies, Inc.) Automated Thawing System runs a protocol simulating a 37° C. water bath. In particular embodiments, once thawed, the vial of cell product can be removed from the automated thawing device.

The step of thawing may be completed within 30 seconds, 1 minute, 2 minutes, 3 minutes, 4 minutes, 5 minutes, 10 minutes, 20 minutes, 30 minutes, 40 minutes, 50 minutes, 1 hour, or more. The step of thawing may be completed within a range of 1 minute to 5 minutes. The step of thawing may be completed within a range of 5 minutes to 10 minutes. The step of thawing may be completed within a range of 10 minutes to 30 minutes. The step of thawing may be completed within a range of 30 minutes to 60 minutes.

The step of thawing may involve warming the cryopreserved cells at a rate of 1° C. per minute, 2° C. per minute, 3° C. per minute, 4° C. per minute, 5° C. per minute, 10° C. per minute, 20° C. per minute, 30° C. per minute, 40° C. per minute, 50° C. per minute, 60° C. per minute, 70° C. per minute, 80° C. per minute, 90° C. per minute, 100° C. per minute, 200° C. per minute, or more. The step of thawing may involve warming the cryopreserved cells at a rate ranging from 1° C. per minute to 5° C. per minute. The step of thawing may involve warming the cryopreserved cells at a rate ranging from 5° C. per minute to 25° C. per minute. The step of thawing may involve warming the cryopreserved cells at a rate ranging from 25° C. per minute to 50° C. per minute. The step of thawing may involve warming the cryopreserved cells at a rate ranging from 50° C. per minute to 100° C. per minute. The step of thawing may involve warming the cryopreserved cells at a rate ranging from 100° C. per minute to 200° C. per minute. The rate of thawing may be continuous, e.g., a constant rate until cells are completely thawed. The rate of thawing may also be discontinuous, e.g., the rate may be more rapid at some temperature ranges relative to the rate at other temperature ranges during thawing, e.g., the rate may be more rapid in the range of −200° C. to 0° C. then in the range of 0° C. to 45° C. during the thawing.

(vi) Preparing Formulations for Administration. The methods of preparing formulations for administration occur after any cryopreservation, genetic modification, cell processing, and/or thawing of the cells. The methods of preparing formulations for administration occur before administering the formulation to the subject.

In particular embodiments, simplified methods for formulation of therapeutic cells for administration to a subject include transferring a volume of cells into a first sterile receptacle to create a cell-filled sterile receptacle; transferring a specified volume of diluent into a second sterile receptacle to create a diluent-filled sterile receptacle; connecting the cell-filled sterile receptacle to the diluent-filled sterile receptacle; mixing the volume of cells with the volume of diluent. In particular embodiments, the methods further include transferring the total volume of cells and diluent into either of the two sterile receptacles to create a product-filled sterile receptacle for administration to the subject.

The product-filled sterile receptacle is filled with a formulation. Formulations described herein can include populations of cells or populations of genetically modified cells suspended in diluent. Formulations can also include media in which cells are disposed, additives, and other diluents. In particular embodiments, formulations are a mixture of cell product and diluent.

This simplified methods are beneficial because it bypasses centrifugation and washing steps as well as eliminates a need for sterile welding and sealing steps. The simplified methods maintain sterility and cell viability while simplifying and speeding up the process for preparing cells for administration.

In particular embodiments, transferring a volume of cells into a first sterile receptacle includes sterilizing an entry port (e.g., septum or cap) of a vial of cell product. In particular embodiments, sterilizing includes applying (e.g. spraying or swabbing) disinfectant. In particular embodiments, disinfectant includes povidone-iodine, isopropyl alcohol, ethanol, methanol, and/or hydrogen peroxide. In particular embodiments, sterilizing includes swabbing the entry port with povidone-iodine and isopropyl alcohol swabs and allowing the entry port to air dry.

In particular embodiments, the volume of cells is calculated to reach a target viable cell concentration. Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells. In formulations disclosed herein, cells are generally in a volume of 10 ml or less, 9 ml or less, 8 ml or less, 7 ml or less, 6 ml or less, 5 ml or less, 4 ml or less, 3 ml or less, 2 ml or less, or 1 ml or less. Hence the target viable cell concentration is typically greater than 10⁴ cells/ml, 10⁷ cells/ml, or 10⁸ cells/ml. In particular embodiments, the target viable cell concentration is 10×10⁶ cells/ml to 325×10⁶ cells/ml.

In particular embodiments, transferring a specified volume of diluent into a second sterile receptacle includes sterilizing the entry port of the container of diluent. In particular embodiments, the diluent includes isotonic solution.

Example isotonic solutions include Normosol®-R (ICU Medical, Inc., Clemente, CA), 0.9% saline, buffered saline, physiological saline, water, Ringer's solution, Lactated Ringer's Solution, Hanks' solution, PLASMA-LYTE A® (Baxter Laboratories, Inc., Morton Grove, IL), Isolyte® (B. Braun Medical, Inc., Bethlehem, PA), or 5% Dextrose in water. In particular embodiments, the isotonic solution includes Normosol®-R (ICU Medical, Inc.). In particular, 100 ml of isotonic solution includes 0-1000 mg sodium chloride, 0-1000 mg sodium acetate, 0-1000 mg sodium gluconate, 0-1000 mg potassium chloride, and 0-1000 mg magnesium chloride hexahydrate. In particular, 100 mL of isotonic solution includes 250-750 mg sodium chloride, 150-400 mg sodium acetate, 250-750 mg sodium gluconate, 10-80 mg potassium chloride, and 10-50 mg magnesium chloride hexahydrate. In 100 mL of Normosol®-R (ICU Medical, Inc.) there is 526 mg sodium chloride, 222 mg sodium acetate, 502 mg sodium gluconate, 37 mg potassium chloride, and 30 mg magnesium chloride hexahydrate. Normosol®-R (ICU Medical, Inc.) can further include HCl and/or NaOH for pH adjustment. Additional isotonic solutions include polyhydric sugar alcohols including trihydric or higher sugar alcohols, such as glycerin, erythritol, arabitol, xylitol, sorbitol, or mannitol.

The volume of diluent required in the methods is dependent on several factors including cell type, methods of cryopreservation, and media in which cells are suspended. The ratio of cells to diluent and/or the volume of diluent required is known by a person skilled in the art and/or can be determined by experimentation. For example, if cells are cryopreserved in DMSO, a dilution ratio of 1:10 v/v of cell volume to diluent volume is sufficient to reduce the remaining concentration of DMSO to levels similar to traditional cell preparation steps (e.g. centrifugation, washing, sterile welding and sealing). In particular embodiments, the specified volume of diluent is at least 1 times, at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times, or at least 15 times the volume of cells. In particular embodiments, the specified volume of diluent is 10 times the volume of cells. In particular embodiments, the specified volume of diluent results in a cell to diluent ratio of 1:1 (v/v), 1:2 (v/v), 1:3 (v/v), 1:4 (v/v), 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v). In particular embodiments, the specified volume of diluent results in a cell to diluent ratio of 1:10 (v/v). In particular embodiments, the specified volume of diluent is a calculated volume. The calculated volume of diluent can vary based on the cryopreservation methods, cryopreservative, concentration of cryopreservative, thawing process, cell concentration, cell type, volume of cell product, and/or isotonic solution.

In particular embodiments, the volume of isotonic solution necessary to perform a 1:10 dilution of the subject cell product is calculated according to the following equations:

$$\begin{matrix}( {T}_{m} \div {C}_{m} ) = T \\ T \div {C}_{V} = Volume to dilute \\ Volume to dilute \times 9 = Volume to diluent\end{matrix}$$

wherein Tₘ is target dose of marker positive cells, Cₘ is the % of marker positive cells, T is the total cells to infuse, and Cy is the concentration of viable cells in cells/mL. In particular embodiments, marker positive cells are EGFRt+ cells.

In particular embodiments, the first and/or second sterile receptacle include any device that retains cells and/or fluid in a sterile environment. It is helpful for the sterile receptacle to have a means for transferring contents such as a plunger or other pressure-based transfer means. In particular embodiments, the first sterile receptacle and/or second sterile receptacle include a syringe, pipette, dropper, injector, or a pressure-based receptacle that allows for the drawing of fluid without compromising sterility. In particular embodiments, the first sterile receptacle is a syringe. In particular embodiments, the second sterile receptacle is a syringe. A syringe is a tube or barrel with a nozzle and piston, plunger, or bulb for sucking in and ejecting liquid or gas. A plunger works by moving linearly though the tube of the syringe such that the contents of the syringe can either be drawn up by creating a vacuum in the syringe or discharged from the syringe through the nozzle.

The nozzle of the sterile receptacle and/or syringe have a connection point. The connection point can be a threaded, barbed, push-fit, twist and lock, compression-based, or crimped. The connection point can be standard such that needles can be fitted to the connection point and fluid dispensing connectors can be fitted to the connection point.

In particular embodiments, a syringe and/or sterile receptacle can be fitted with a needle. In particular embodiments, the needle is a safety-shielded needle. In particular embodiments, the needle is a 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, or a 27 gauge needle. In particular embodiments, the needle is an 18 gauge needle. In particular embodiments, if the sterile receptacle is a syringe, the mixing includes pushing the plunger of the syringe. In other embodiments, mixing includes causing flow between the fluid dispensing connector and sterile receptacles such that the contents become substantially homogenously mixed.

In particular embodiments, connecting the cell-filled sterile receptacle to the diluent-filled sterile receptacle includes removing any needles from the cell-filled sterile receptacle and/or the diluent-filled sterile receptacle and connecting a fluid dispensing connector to both sterile receptacles. In particular embodiments, the fluid dispensing connector includes any means having at least two ports by which fluid is transferred between at least two receptacles connected to the ports in sterile manner. In particular embodiments, a fluid dispensing connector includes sterile tubing or a sterile fluid lock between sterile receptacles. In particular embodiments, the fluid dispensing connector includes at least two ports. In particular embodiments, the cell-filled sterile receptacle is connected to a first port on the fluid dispensing connector and the diluent-filled sterile receptacle is connected to a second port on the fluid dispensing connector.

A fluid dispensing connector is any conduit, pipe, hose, or tube through which a fluid can flow into or out of a connected component. The fluid can be in an open-channel or it can be a pressurized fluid. The fluid dispensing connector can connect to one component or a plurality of components. The fluid dispensing connector can have channels that can be closed or open, soft or rigid, or contain valves. The fluid dispensing connector can connect to components through any means known in the art including a threaded connection, suction, push-in fittings, twist and lock, cap fittings, compression fittings, tension (or compression) fittings, or crimped fittings. Fluid dispensing connectors include FDC-1000 Fluid Dispensing Connector (B-Braun Medical, Inc., Melsungen, Germany), Vygon Gluid Dispensing Connector (SKU: VYAMS1200-BX), Baxter Rapidfill Connector-H93813701 (Deerfield, IL), luer lock connectors.

In particular embodiments, mixing the volume of cells with the volume of diluent includes transferring the contents of the cell-filled sterile receptacle and diluent-filled sterile receptacle through the fluid dispensing connector such that the cell product and diluent form a mixture. If the sterile receptacle is a syringe with a plunger, mixing can be accomplished by compressing the plunger of each syringe consecutively such that the contents of the syringe flows into and out of each syringe. The plungers would be consecutively compressed a plurality of times until the contents of the two syringes is thoroughly mixed.

Mixing refers to combining two or more substances, elements, or things into one mass, collection, or assemblage. A mixture refers to a composition of made up of two or more substances that are physically combined, not chemically combined, and are capable of being separated. In particular embodiments, a mixture includes a volume containing cells and diluent. In particular embodiments, the cells are evenly distributed throughout the diluent in a mixture.

In particular embodiments, the total volume of cell product and diluent is transferred to either sterile receptacle to produce a product-filled sterile receptacle. In particular embodiments, the total volume of cells and diluent is less than 15 ml, less than 14 ml, less than 13 ml, less than 12 ml, less than 11 ml, less than 10 ml, less than 9 ml, less than 8 ml, less than 7 ml, less than 6 ml, less than 5 ml, less than 4 ml, less than 3 ml, less than 2 ml, or less than 1 ml. In particular embodiments, the total volume of cells and diluent is less than 5 ml.

In particular embodiments, the fluid dispensing connector is disconnected from the product-filled sterile receptacle. In particular embodiments, the product-filled sterile receptacle is plugged with a luer plug. In particular embodiments, the product-filled sterile receptacle is transferred to a clinical setting for administration to a subject.

Formulations can be prepared for administration by, e.g., injection or infusion. The formulations and compositions can further be formulated for intravenous, intraarterial, bone marrow, intradermal, intranodal, intralymphatic, intraperitoneal, intralesional, intraprostatic, intravaginal, intrarectal, topical, intrathecal, intratumoral, intramuscular, intravesicular, and/or subcutaneous injection. In particular embodiments, the administration includes infusion.

More specifically, in particular embodiments, methods for formulation of therapeutic cells for administration to a subject may include thawing a vial of cryopreserved cells using an automated thawing device to create a vial of cell product; once thawed, disinfecting the entry port of the vial by swabbing it with povidone-iodine and isopropyl alcohol swabs and allowing the entry port to air dry; fitting a first syringe with a first 18 gauge safety shielded needle; piercing the entry port of the vial of cell product with the first needle; withdrawing the specified volume of cell product into the first syringe to create a cell-filled syringe; disinfecting the entry port of the container of diluent by swabbing it with povidone-iodine and isopropyl alcohol swabs and allowing the entry port to air dry; fitting a second syringe with a second 18 gauge safety shielded needle; piercing the entry port of the container of diluent with the second needle; withdrawing the specified volume of diluent into the second syringe to create a diluent-filled syringe, wherein the specified volume of diluent; removing said first needle on the cell-filled syringe and attaching the cell-filled syringe to a first port on a fluid dispensing connector having a first and second port; removing said second needle on the diluent-filled syringe and connecting the diluent-filled syringe to the second port of the fluid dispensing connector; mixing the cell product and diluent between the syringes and fluid dispensing connector; transferring all of the volume of cell product and diluent into one of the two connected syringes creating a product-filled syringe. In particular embodiments, the methods for formulation can further include removing the fluid dispensing connector from the product-filled syringe; and placing a luer plug on the product-filled syringe. In particular embodiments, the product-filled syringe is transferred to a clinical setting for administration to a subject.

In addition to cell product and diluent, formulations can additionally include pharmaceutically acceptable carriers.

The phrase “pharmaceutically acceptable” refer to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

Depending on the context and active compound for delivery, “pharmaceutically acceptable carriers” includes any adjuvant, excipient, glidant, diluent, preservative, dye/colorant, surfactant, wetting agent, dispersing agent, suspending agent, stabilizer, isotonic solution (or isotonic agent), solvent, surfactant, or emulsifier which meets the requirements noted above. Exemplary pharmaceutically acceptable carriers are disclosed in Remington's Pharmaceutical Sciences, 18th Ed. Mack Printing Company, 1990. Moreover, formulations and compositions can be prepared to meet sterility, pyrogenicity, general safety, and purity standards as required by the US FDA Office of Biological Standards and/or other relevant foreign regulatory agencies.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

In particular embodiments, formulations can include one or more cell types or one or more genetically modified cell types (e.g., modified T cells, NK cells, or stem cells). The different populations of genetically modified cells can be provided in different ratios.

(vii) Methods of Use. The methods disclosed herein are to simplify the preparation of cells for administration to a subject. The simplified methods maintain the sterility and viability of cells while reducing the number of steps and time needed to prepare the cells. After preparation of the cells into a formulation for administration, the formulation can be used for treating subjects (humans, veterinary animals (dogs, cats, reptiles, birds, etc.) livestock (horses, cattle, goats, pigs, chickens, etc.) and research animals (monkeys, rats, mice, fish, etc.). Treating subjects includes delivering therapeutically effective amounts. Therapeutically effective amounts include those that provide effective amounts, prophylactic treatments and/or therapeutic treatments without undue toxicity.

In particular embodiments, the subject is human. In particular embodiments, the subject is greater than 1 years old. In particular embodiments, the subject is greater than 1 year old, greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, greater than 16 years old, greater than 17 years old, greater than 18 years old, greater than 19 years old, greater than 20 years old, greater than 21 years old, greater than 22 years old, greater than 23 years old, greater than 24 years old, greater than 25 years old, or greater than 26 years old. In particular embodiments, the subject is less than 26 years old.

An “effective amount” is the amount of a formulation or cell product necessary to result in a desired physiological effect. Effective amounts are often administered for research purposes. Effective amounts disclosed herein can cause an intended physiological effect in a research model. For example, an effective amount can cause cell killing after administration in a research model. In other examples, an effective amount can cause hematopoietic engraftment in a research model.

A “prophylactic treatment” includes a treatment administered to a subject who does not display signs or symptoms of a condition (e.g., cancer or an infection) or displays only early signs or symptoms of the condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

Therapeutically effective amounts can be achieved by administering single or multiple doses during the course of a treatment regimen (e.g., daily, every other day, every 3 days, weekly, every 2 weeks, monthly, every 2 months, every 4 months, every 6 months, yearly, etc.). In particular embodiments, several courses of treatment can be administered. In particular embodiments, 7, 6, 5, 4, 3, 2, or 1 course of treatment is administered.

As indicated, the formulations can be administered by injection, transfusion, implantation or transplantation. In particular embodiments, formulations and compositions are administered parenterally. The phrases “parenteral administration” and “administered parenterally” refer to modes of administration other than enteral and topical administration, usually by injection, and includes, intravascular, intravenous, intramuscular, intraarterial, intrathecal, intracapsular, intratumoral, intraperitoneal, and subcutaneous, injection and infusion. In particular embodiments, the formulations described herein are administered to a subject by infusion. In particular embodiments, the formulations described herein are administered to a subject by injection at the site of tumor resection. In particular embodiments, the formulations described herein are administered to a subject by injection into the ventricular system of the central nervous system.

Cancers that can be treated by formulations disclosed herein include: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B cell lymphoma, T cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary cancers that can be treated according to the disclosure include hematopoietic tumors of lymphoid lineage, for example T cell and B cell tumors, including: T cell disorders such as T-prolymphocytic leukemia (T-PLL), including of the small cell and cerebriform cell type; large granular lymphocyte leukemia (LGL) of the T cell type; Sezary syndrome (SS); adult T cell leukemia lymphoma (ATLL); hepatosplenic T cell lymphoma; peripheral/post-thymic T cell lymphoma (pleomorphic and immunoblastic subtypes); angioimmunoblastic T cell lymphoma; angiocentric (nasal) T cell lymphoma; anaplastic (Ki 1+) large cell lymphoma; intestinal T cell lymphoma; and T-lymphoblastic lymphoma/leukemia (T-Lbly/T-ALL). In particular embodiments, formulations disclosed herein are used to treat central nervous system tumor, glioma, ependymoma, medulloblastoma, germ cell tumor, atypical teratoid, primitive neuroectodermal tumor, choroid plexus carcinoma, or pineoblastoma.

When administered cells are genetically-modified, they can be modified to express a therapeutic molecule with ligand binding domains that bind the following exemplary cancer antigens selected based on the cancer experienced by a subject: bladder cancer antigens: MUC16, PD-L1, EGFR; breast cancer antigens: HER2, ERBB2, ROR1, PD-L1, EGFR, MUC16, FOLR, CEA; cholangiocarcinoma antigens: mesothelin, PD-L1, EGFR; colorectal cancer antigens: CEA, PD-L1, EGFR; glioblastoma antigens: EGFR variant III (EGFRvIII), IL13Ra2; lung cancer antigens: ROR1, PD-L1, EGFR, mesothelin, MUC16, FOLR, CEA, CD56; Merkel cell carcinoma antigens: CD56, PD-L1, EGFR; mesothelioma antigens: mesothelin, PD-L1, EGFR; neuroblastoma antigens: ROR1, glypican-2, CD56, disialoganglioside, PD-L1, EGFR; ovarian cancer antigens: EpCam, L1-CAM, MUC16, folate receptor (FOLR), Lewis Y, ROR1, mesothelin, WT-1, PD-L1, EGFR, CD56; melanoma antigens: Tyrosinase related protein 1 (TYRP1/gp75); GD2, PD-L1, EGFR; multiple myeloma antigens: B-cell maturation antigen (BCMA), PD-L1, EGFR; pancreatic cancer antigens: mesothelin, CEA, CD24, ROR1, PD-L1, EGFR, MUC16; prostate cancer antigens: PSMA, WT1, Prostate Stem Cell antigen (PSCA), SV40 T, PD-L1, EGFR; renal cell carcinoma antigens: carboxy-anhydrase-IX (CAIX); PD-L1, EGFR; and stem cell cancer antigens: CD133, PD-L1, EGFR. Other examples are known to those of ordinary skill in the art. In particular embodiments, central nervous system tumor antigens are targeted with Her2-specific therapeutic molecules. In particular embodiments, anti-Her2 therapeutic molecules are used to target antigens on cancer cells representing central nervous system tumors, glioma, ependymoma, medulloblastoma, germ cell tumor, atypical teratoid (rhabdoid tumor), primitive neuroectodermal tumor, choroid plexus carcinoma, and/or pineoblastoma.

In certain examples, a cancerous sample from a subject can be characterized for the presence of certain biomarkers or cell surface markers. For example, breast cancer cells from a subject can be positive or negative for each of Her2Neu, Estrogen receptor, and/or the Progesterone receptor. A tumor antigen or cell surface molecule that is found on the individual subject's tumor cells as well as a therapeutic molecule with a binding domain that binds the antigen is selected.

In particular embodiments, therapeutically effective amounts of formulations provide anti-cancer effects. Anti-cancer effects include a decrease in the number of malignant cells, decrease in the number of metastases, a decrease in tumor volume, an increase in life expectancy, induced chemo- or radio-sensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

Infections that can be treated by disclosed formulations include bacterial, viral, fungal, parasitic, and arthropod infections. In particular embodiments, the infections are chronic. In particular embodiments, bacterial infections can include infections caused by Staphylococcus spp., Streptococcus spp., Campylobacter jejuni, Clostridium botulinum, Clostridium difficile, Escherichia coli, Listeria monocytogenes, Salmonella, Vibrio, Chlamydia trachomatis, Neisseria gonorrhoeae, and Treponema pallidum. In particular embodiments, viral infections can include infections caused by rhinovirus, influenza virus, respiratory syncytial virus (RSV), coronavirus, herpes simplex virus-1 (HSV-1), varicella-zoster virus (VZV), hepatitis A, norovirus, rotavirus, human papillomavirus (HPV), hepatitis B, human immunodeficiency virus (HIV), herpes simplex virus-2 (HSV-2), Epstein-Barr virus (EBV), West Nile virus (WNV), enterovirus, hepatitis C, human T-lymphotrophic virus-1 (HTLV-1), and Merkel cell polyomavirus (MCV). In particular embodiments, fungal infections can include infections caused by Trychophyton spp. and Candida spp. In particular embodiments, parasitic infections can include infections caused by Giardia, toxoplasmosis, E. vermicularis, Trypanosoma cruzi, Echinococcosis, Cysticercosis, Toxocariasis, Trichomoniasis, and Amebiasis. In particular embodiments, arthropod infections can include infections spread by arthropods infected with viruses or bacteria, including California encephalitis, Chikungunya, dengue, Eastern equine encephalitis, Powassan, St. Louis encephalitis, West Nile, Yellow Fever, Zika, Lyme disease, and babesiosis.

In particular embodiments, therapeutically effective amounts of formulations provide anti-infection effects. Anti-infection effects include a decrease in: the amount or level of infective pathogen, fatigue, loss of appetite, weight loss, fevers, night sweats, chills, aches and pains, diarrhea, bloating, abdominal pain, skin rashes, coughing, and/or a runny nose.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of disease, stage of disease, previous or concurrent therapeutic interventions, idiopathy of the subject and route of administration.

Therapeutically effective amounts of formulations to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells in diluent.

In addition to administration to a subject, the methods and kits described herein can be used for any appropriate downstream application, e.g., research, drug discovery, biologics production, etc.

(viii) Kits. Kits to perform the simplified methods described herein are also provided. The kits may include material(s), which may be desirable from a user standpoint, such as at least two sterile receptacles and a fluid dispensing connector. The kits can further include a needle, a diluent, a disinfectant, and/or a Luer plug. Furthermore, in particular embodiments, the kits can include materials useful for cell harvesting, cryopreservation, genetic modification, thawing, diluting, administration and/or any other step required to provide cells to a subject. These additional materials may include a cryopreservative or a vial.

The kits according to the present disclosure may also include instructions for carrying out the methods. Instructions included in the kits of the present disclosure may be affixed to packaging material or may be included as a package insert. While instructions are typically written or printed materials, they are not limited to such. Any medium capable of storing such instructions and communicating them to an end user is contemplated by this disclosure. Such media include electronic storage media (e.g., magnetic discs, tapes, cartridges, chips), optical media (e.g., CD ROM), and the like. As used herein, the term “instructions” can include the address of an internet site which provides instructions.

The components of the kits may be provided in a single container, e.g., a plastic or styrofoam box, in relatively close confinement. Typically, the kits are conveniently packaged for use by a health care professional. In certain embodiments, the components of the kits are sterilely packaged for use in a sterile environment such as an operating or treatment room or physician's office.

### (ix) Exemplary Embodiments

1. A method of preparing a therapeutic cell product for administration to a subject including transferring a volume of therapeutic cells into a first sterile receptacle to create a cell-filled sterile receptacle;
transferring a specified volume of diluent into a second sterile receptacle to create a diluent-filled sterile receptacle;
connecting the cell-filled sterile receptacle to the diluent-filled sterile receptacle with a fluid dispensing connector; and
mixing the volume of cells with the volume of diluent thereby preparing the therapeutic cell product for administration to the subject.

2. The method of embodiment 1, wherein the first sterile receptacle is a syringe.

3. The method of embodiments 1 or 2, wherein the second sterile receptacle is a syringe.

4. The method of embodiments 2 or 3, wherein the syringe is fitted with a needle.

5. The method of embodiment 4, wherein the needle includes a safety-shielded needle.

6. The method of embodiments 4 or 5, wherein the needle includes a 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, or a 27 gauge needle.

7. The method of any of embodiments 4-6, wherein the needle includes an 18 gauge safety-shielded needle.

8. The method of any of embodiments 4-7, wherein the needle is removed before connecting the cell-filled sterile receptacle to the diluent-filled sterile receptacle with a fluid dispensing connector.

9. The method of any of embodiments 1-8, wherein transferring the volume of cells into the first sterile receptacle includes drawing cells into the first sterile receptacle.

10. The method of any of embodiments 1-9, wherein the volume of cells includes a cell density ranging from 10×10⁶ cells/ml to 325×10⁶ cells/ml.

11. The method of any of embodiments 1-10, wherein transferring the specified volume of diluent into the second sterile receptacle includes drawing diluent into the second sterile receptacle.

12. The method of any of embodiments 1-11, wherein a ratio of volume of cells to specified volume of diluent is selected from 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v).

13. The method of any of embodiments 1-12, wherein a ratio of volume of cells to specified volume of diluent is 1:10 (v/v).

14. The method of embodiment 13, wherein the specified volume of diluent is at least 4 times, at least 5 times, at least 6 times, at least 7 times, at least 8 times, at least 9 times, at least 10 times, at least 11 times, at least 12 times, at least 13 times, at least 14 times, or at least 15 times the volume of cells.

15. The method of embodiments 13 or 14, wherein the specified volume of diluent is 9 times the volume of cells.

16. The method of any of embodiments 1-15, wherein the diluent includes an isotonic solution.

17. The method of embodiment 16, wherein the isotonic solution includes less than 10 mg/mL sodium chloride, less than 10 mg/mL sodium acetate, less than 10 mg/mL sodium gluconate, less than 10 mg/mL potassium chloride, and less than 10 mg/mL magnesium chloride hexahydrate.

18. The method of embodiments 16 or 17, wherein the isotonic solution includes 2.5-7.5 mg/mL sodium chloride, 1.5-4.0 mg/mL sodium acetate, 2.5-7.5 mg/mL sodium gluconate, 0.1-0.8 mg/mL potassium chloride, and 0.1-0.5 mg/mL magnesium chloride hexahydrate.

19. The method of any of embodiments 16-18, wherein the isotonic solution includes 5.26 mg/mL sodium chloride, 2.22 mg/mL sodium acetate, 5.02 mg/mL sodium gluconate, 0.37 mg/mL potassium chloride, and 0.30 mg/mL magnesium chloride hexahydrate.

20. The method of any of embodiments 16-19, wherein the isotonic solution further includes HCl for pH adjustment.

21. The method of any of embodiments 16-20, wherein the isotonic solution further includes NaOH for pH adjustment.

22. The method of any of embodiments 1-21, wherein the fluid dispensing connector includes at least 2 ports.

23. The method of embodiment 22, wherein the fluid dispensing connector includes a first port and a second port.

24. The method of embodiment 23, wherein the cell-filled sterile receptacle is attached to the first port on the fluid dispensing connector and the diluent-filled sterile receptacle is attached to the second port on the fluid dispensing connector.

25. The method of any of embodiments 1-24, wherein mixing the volume of cells with the volume of diluent includes forcing cells and diluent to flow between the cell-filled sterile receptacle, fluid dispensing connector, and diluent-filled sterile receptacle to create a total mixed volume of cells and diluent.

26. The method of any of embodiments 1-25, wherein the first sterile receptacle includes a first syringe, the second sterile receptacle includes a second syringe, and the mixing includes pushing a plunger on the first syringe and the second syringe consecutively and repetitively such that the volume of cells and volume of diluent mix to create a total mixed volume of cells and diluent.

27. The method of embodiments 25 or 26, wherein the total mixed volume is less than 10 mL, less than 9 mL, less than 8 mL, less than 7 ml, less than 6 mL, less than 5 mL, less than 4 mL, less than 3 mL, less than 2 mL, or less than 1 mL.

28. The method of embodiment 27, wherein the total mixed volume is less than 5 mL.

29. The method of any of embodiments 25-28, including transferring the total mixed volume into either the cell-filled sterile receptacle or the diluent-filled sterile receptacle to create a product-filled sterile receptacle.

30. The method of embodiment 29, including transferring the total mixed volume into the cell-filled sterile receptacle to create the product-filled sterile receptacle.

31. The method of embodiment 29, including transferring the total mixed volume into the diluent-filled sterile receptacle to create the product-filled sterile receptacle.

32. The method of any of embodiments 29-31, including removing the product-filled sterile receptacle from the fluid dispensing connector.

33. The method of any of embodiments 29-32, including plugging the product-filled sterile receptacle with luer plug.

34. The method of any of embodiments 29-33, including storing the product-filled sterile receptacle in a vessel on dry ice.

35. The method of any of embodiments 29-34, including transferring the product-filled sterile receptacle to a clinic for administration to the subject.

36. The method of any of embodiments 1-35, wherein the subject has cancer.

37. The method of embodiment 36, wherein the cancer is a central nervous system tumor, glioma, ependymoma, medulloblastoma, germ cell tumor, atypical teratoid, primitive neuroectodermal tumor, choroid plexus carcinoma, or pineoblastoma.

38. The method of embodiments 36 or 37, wherein the cancer is a central nervous system tumor.

39. The method of any of embodiments 1-38, wherein the therapeutic cells include cells from the subject.

40. The method of embodiment 39, wherein the cells from the subject include immune cells.

41. The method of embodiment 40, wherein the immune cells include T cells.

42. The method of embodiment 41, wherein the T cells are CD4+ T cells.

43. The method of embodiments 41 or 42, wherein the T cells are CD8+ T cells.

44. The method of any of embodiments 41-43, wherein the T cells are CD4+ and CD8+ T cells.

45. The method of any of embodiments 41-44, wherein the T cells are genetically modified to express a therapeutic molecule.

46. The method of embodiment 45, wherein the therapeutic molecule includes a chimeric antigen receptor (CAR).

47. The method of any of embodiments 41-46, wherein the T cells are genetically modified to express a CAR and EGFRt.

48. The method of embodiments 46 or 47, wherein the CAR is an anti-Her2 CAR.

49. The method of any of embodiments 1-48, further including administering the therapeutic cell product to the subject.

50. The method of embodiment 49, wherein the administering includes injection, infusion, implantation, or transplantation.

51. The method of embodiments 49 or 50, wherein the administering includes injecting the therapeutic cell product at the site of a tumor resection in the subject.

52. The method of any of embodiments 49-51, wherein the administering includes injecting the therapeutic cell product into the ventricular system of the central nervous system of the subject.

53. The method of embodiments 49 or 50, wherein the administering includes infusion.

54. The method of any of embodiments 1-53, wherein the subject is greater than 1 year old, greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, greater than 16 years old, greater than 17 years old, greater than 18 years old, greater than 19 years old, greater than 20 years old, greater than 21 years old, greater than 22 years old, greater than 23 years old, greater than 24 years old, or greater than 25 years old.

55. The method of any of embodiments 1-54, wherein the subject is greater than 1 year old.

56. The method of any of embodiments 1-55, wherein the subject is less than 26 years old.

57. The method of any of embodiments 1-55, wherein the subject is greater than 26 years old.

58. The method of any of embodiments 1-57, wherein the method does not include centrifugation.

59. The method of any of embodiments 1-58, wherein the method does not include sterile welding.

60. The method of any of embodiments 1-59, wherein the method does not include a wash step.

61. The method of any of embodiments 1-60, wherein the method further includes cryopreserving the therapeutic cells to create cryopreserved cells and thawing the cryopreserved cells to create thawed therapeutic cells before transferring the volume of thawed therapeutic cells into the first sterile receptacle.

62. The method of embodiment 61, wherein the cryopreserving includes suspending the therapeutic cells in a freezing medium and reducing the temperature to a cryopreservation temperature to create cryopreserved cells.

63. The method of embodiment 62, wherein the freezing medium includes a cryopreservative.

64. The method of embodiment 63, wherein the cryopreservative is DMSO.

65. The method of any of embodiments 62-64, wherein the freezing medium includes 5% DMSO.

66. The method of any of embodiments 62-65, wherein the cryopreservation temperature ranges from −205° C. to −60° C.

67. The method of any of embodiments 62-66, wherein the cryopreserved cells are stored in liquid nitrogen.

68. The method of any of embodiments 61-67, wherein the thawing includes incubating the cryopreserved cells at progressively increasing temperatures.

69. The method of embodiment 68, wherein the incubating includes putting the cryopreserved cells in a temperature controlled incubator.

70. The method of embodiment 69, wherein the temperature controlled incubator includes a controlled water bath, a temperature controlled oven, or an automated thawing device.

71. The method of any of embodiments 61-70, wherein the thawing includes using an automated thawing device.

72. The method of any of embodiments 61-71, wherein the thawing includes incubating the cryopreserved cells at a temperature ranging from 15° C. to 30° C.

73. The method of any of embodiments 61-71, wherein the thawing includes incubating the cryopreserved cells at a temperature ranging from 30° C. to 45° C.

74. The method of any of embodiments 61-73, wherein the thawing includes incubating the cryopreserved cells at a temperature of 37° C.

75. The method of any of embodiments 61-74, further including determining a viability of the thawed therapeutic cells.

76. The method of embodiment 75, wherein the viability of thawed therapeutic cells is greater than 65%.

77. The method of embodiments 75 or 76, wherein the viability of thawed therapeutic cells is greater than 70%.

78. A method for formulation of therapeutic cells for administration to a subject including thawing cryopreserved cells to create thawed cells; transferring a volume of thawed cells into a first syringe to create a cell-filled syringe; transferring a specified volume of diluent into a second syringe to create a diluent-filled syringe; connecting the cell-filled syringe to the diluent-filled syringe with a fluid dispensing connector; mixing the volume of cells with the volume of diluent to create a total mixed volume; and transferring the total mixed volume into either the cell-filled syringe or the diluent-filled syringe.

79. The method of embodiment 78, wherein the thawing includes using an automated thawing device.

80. The method of embodiments 78 or 79, including a ratio of volume of thawed cells to volume of diluent of 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v).

81. The method of any of embodiments 78-80, including a ratio of volume of thawed cells to volume of diluent of 1:10 (v/v).

82. The method of any of embodiments 78-81, wherein the total mixed volume is transferred to the cell-filled syringe.

83. The method of any of embodiments 78-82, including disconnecting the fluid dispensing connector from the cell-filled syringe and plugging the cell-filled syringe with a luer plug.

84. The method of any of embodiments 78-83, wherein the total mixed volume is transferred to the diluent-filled syringe.

85. The method of any of embodiments 78-84, including disconnecting the fluid dispensing connector from the diluent-filled syringe and plugging the diluent-filled syringe with a luer plug.

86. A kit for dilution of cell product including syringes, needles, fluid dispensing connector, and diluent.

87. The kit of embodiment 86, including at least two syringes.

88. The kit of embodiments 86 or 87, wherein the needles attach to the syringes.

89. The kit of any of embodiments 86-88, wherein the needles include 18 gauge safety-shielded needles.

90. The kit of any of embodiments 86-89, wherein the fluid dispensing connector attaches to the syringes.

91. The kit of any of embodiments 86-90, wherein the diluent includes an isotonic solution.

92. The kit of embodiment 91, wherein the isotonic solution includes 5.26 mg/mL sodium chloride, 2.22 mg/mL sodium acetate, 5.02 mg/mL sodium gluconate, 0.37 mg/ml potassium chloride, and 0.30 mg/mL magnesium chloride hexahydrate.

93. The kit of any of embodiments 86-92, including NaOH and/or HCl for pH adjustment.

94. The kit of any of embodiments 86-93, including a luer plug.

95. The kit of any of embodiments 86-94, including disinfectant.

96. The kit of embodiment 95, wherein the disinfectant includes povidone-iodine, isopropyl alcohol, ethanol, methanol, and/or hydrogen peroxide.

97. The kit of embodiments 95 or 96, wherein the disinfectant includes povidone-iodine and/or isopropyl alcohol swabs.

98. The kit of any of embodiments 86-97, including instructions for dilution of cell product.

(x) Experimental Examples. Traditionally, autologous CAR T Cell products are manufactured and stored in cryopreserved form. Prior to infusion into the subject, cryopreserved CAR T Cell Products are taken through Thaw-and-Wash and final formulation steps and a viability of ≥70% of the final formulated CAR T cell product is required for its infusion.

In the case of an Out of Specification (OOS) result observed during final release testing of the manufactured cell product, it is standard practice to contact the FDA and seek approval for the release of the OOS cell product. However, in the case of OOS products following a thaw/wash procedure, due to the short time period (90 minutes) between cell thaw and expiration, it is not always possible to obtain approval from the FDA to allow for infusion of the formulated product before it expires. Given the refractory progressive disease state of the study subjects as well as the limited supply of available doses, revised release criteria for infusion of the final formulated product includes:
(a) should a cell product's viability lie between 65-70% (within the error limit of the cell counting process) following the final formulation preparation process, a designee will be able to assess and allow the cell product to be infused without consulting the FDA. The Sponsor will file documentation pertaining to the planned protocol deviation in the study files.
(b) should a cell product's viability lie below 65% following final formulation preparation process, the cell product will be discarded, and a new cryopreserved dose will be thawed to proceed through the final formulation preparation.

The revised release criteria were based on the perspective that there would be minimal risk to the subject receiving the cell product that is within 5% of the specified viability criteria (>70%).

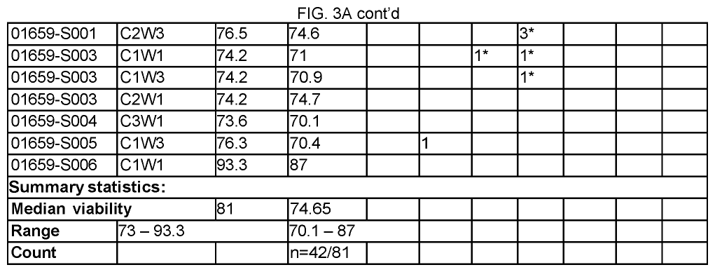

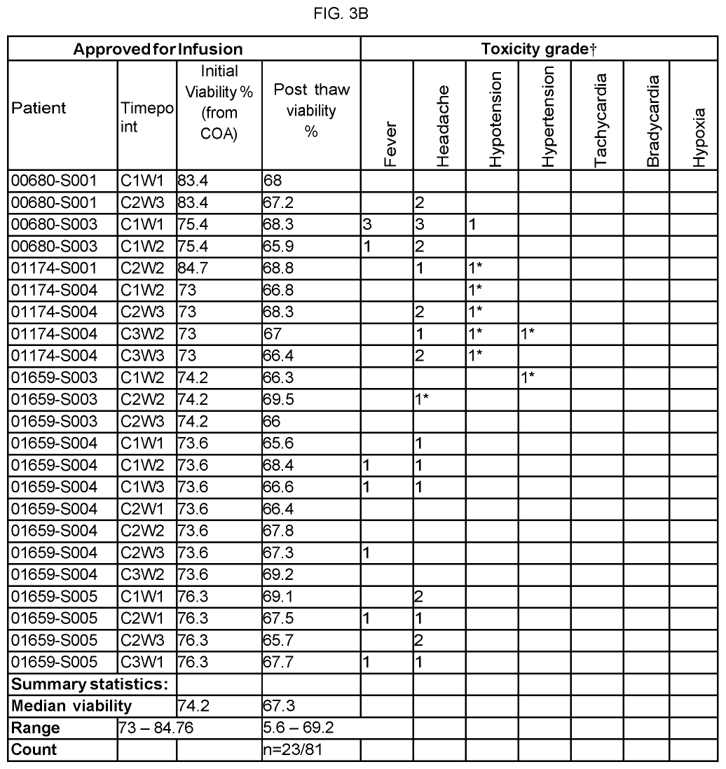

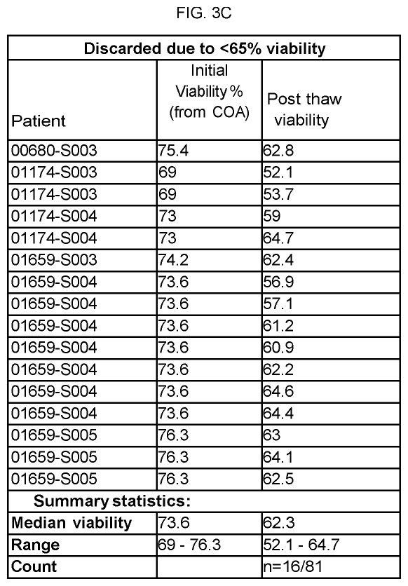

Since the implementation of the revised cell product release criteria, 20% of vials have been discarded due to viability <65% (52.1-64.7%) and an additional 28% were infused but required Sponsor approval due to viability 65-<70% (65.6-69.2%) (FIGS. 3A-3C). This occurred across all trials IND 017857, IND 018234 and IND 019043 although there are too few patients to evaluate whether there is any difference in incidence across the trials. No patient developed infusion related toxicity during the administration or within the immediate 6 hours of infusion. Moreover, there have been no observed dose limiting toxicities and the spectrum of toxicities occurring within 7 days of the investigational product including transient symptoms and signs of local inflammation was similar for products of >70% viability or those 65-<70% viability (FIGS. 3A-3C).

Based on these observations, a thaw and dilute process was developed to replace the current thaw and wash process for preparation of infusion cell doses.

In order to qualify this process, analytical testing was performed to determine the amount of DMSO remaining following processing of representative cryopreserved CAR T cell products by both the currently approved methods and the disclosed thaw/dilute process. Supernatants were collected from CAR T cell products and sent to Analytic Resource Laboratories, Lehi, Utah for DMSO analysis. As depicted in the table below, thawing and diluting cell products at a 1:10 dilution in Normasol achieved DMSO concentrations equivalent to the standard thaw and wash process (Table 1).

TABLE 1
DMSO Concentrations in Thaw-and-Wash
vs. Thaw-and-Dilute Cell Products.

| | Percent (%) DMSO Observed |
|---|---|
| Normasol alone | <0.1 |
| CS-5 Cryo Solution (unmanipulated) | 5.8 |

| | Cell Product | Cell Product | Cell Product |
|---|---|---|---|
| | #1 | #2 | #3 |
| Thaw/Wash Protocol | | | |
| 1^{st} wash supernatant | 0.8 | 0.8 | 1.0 |
| 2^{nd} wash supernatant | 0.5 | 0.5 | 0.6 |
| Final product supernatant | 0.5 | 0.6 | 0.6 |
| Thaw/ Dilute Protocol | | | |
| 1:2 dilution supernatant | 2.8 | 2.9 | 2.9 |
| 1:6 dilution supernatant | 0.9 | 0.9 | 0.9 |
| 1:10 dilution supernatant | 0.6 | 0.6 | 0.7 |

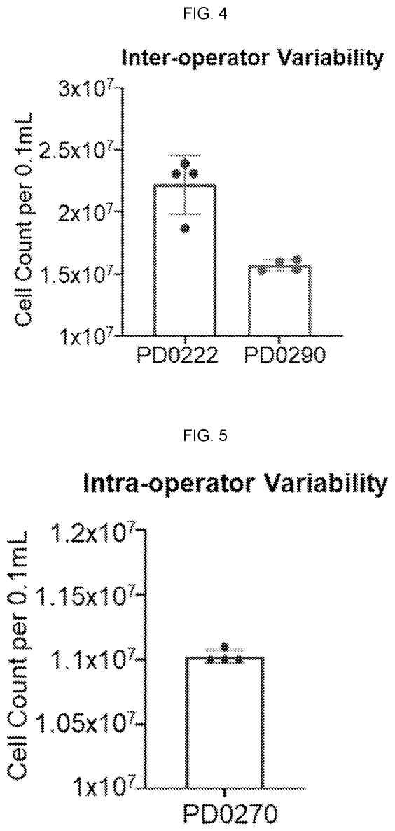

Knowing that the concentration of cryopreserved CAR T cells needs to increase so as to be able to perform a 1:10 dilution upon thaw and not exceed a volume of 5 mL for subject infusion an experiment was performed to look at the reproducible precision of those dilutions when starting with a very small volume of highly concentrated cells. To determine inter-operator precision of cell counts four individuals each thawed 2 concentrated CAR T cell products and performed cell counts after making a 1:10 dilution in Normasol (FIG. 4).

Additionally, one individual performed multiple 1:10 dilutions of a thawed CAR T product to determine intra-operator precision (FIG. 5).

These experiments conclude that by concentrating cryopreserved CAR T products and performing a 1:10 dilution in Normasol for dosing upon thaw allows for infusing a precise dose of CAR T cells with an equivalent percentage of residual DMSO compared to the current thaw and wash procedure. Moreover, the method substantially reduces the loss of products that would otherwise fail due to poor viability and recovery observed as a result of the thaw and wash process.

The volume required for dose administration will continue to be determined based on the viable cell recovery of a representative aliquot of the final cell product during required release testing.

### Procedure.

1. The thaw and dilute procedure is completed, and the transfer of custody is performed with clinical staff at bedside for immediate administration.

2. Determination of Volumes and Label Preparation:
2.1 Determine volume for cell suspension and volume of diluent using Attachment 1 below.

| Attachment 1: Calculation of Infusion Volume | | | | | |
|---|---|---|---|---|---|
| Data Entry Section VI: Final Formulation | | | | | Line |
| _{——}_{——} | ÷ | _{——}_{——}% | = | TNC to | 1 |
| Target Dose EGFRt+ | | EGFRt+ | | Infuse | |
| Cells from | | from CofA | | | |
| PRF/Medication | | | | | |
| Order | | | | | |
| _{——}_{——} | ÷ | _{——}_{——} | = | _{——}_{——}mL | 2 |
| TNC to Infuse | | ×10⁶ | | Volume to | |
| from Line 1 | | cells/mL | | dilute | |
| | | Viable | | (rounded | |
| | | conc. | | to tenths) | |
| | | from CofA | | | |
| _{——}_{——}mL | × | 9 | = | _{——}_{——}mL | 3 |
| Volume to dilute | | | | Volume of | |
| (rounded to tenths) | | | | Diluent | |

Infusion Product Information

| | |
|---|---|
| Date/Time of Thaw: | Date/Time of Expiration: |
| | (+4 hrs) |
| Volume in syringe (mL): | Number of EGFRt+ cells in |
| | syringe: |

3. Final preparation prior to Thaw:
3.1. Reset and program the data logger for data collection before use. Place the data logger in the “Golden Hour” insulated container. Refer to SOP EQ-050, “Operation and Maintenance of the TCTemp 2000 Data Logger” for details.

4. Thaw and Dilution:
4.1. Transfer the following to the BSC:
   4.1.1. Normosol
   4.1.2. Plasma Transfer Set
   4.1.3. Syringe (3 mL or 5 mL)
   4.1.4. FDC
   4.1.5 Luer plug
4.2. Close the roller clamp on a plasma luer transfer set and spike it into a bag of Normosol.
4.3. Connect the syringe to the luer end of the plasma transfer set spiked into bag of Normosol and fill syringe to the appropriate “Diluent volume”.
4.4. Close clamp on Normosol.
4.5. Remove syringe and cap with female end of luer plug.

5. Thaw CellSeal vial using auto-thawing system
NOTE: For all following steps, ensure the CellSeal vial remains upright and that no liquid comes in contact with the filter.
5.1. Plug in the CellSeal Automated Thawing (CAT) device to turn the unit on. For more information, refer to EQ-055 “Operation and Maintenance of CellSeal Automated Thawing System.”
5.2. Select the “2 mL vial @ 1.5 mL: R” profile.
   5.2.1. The indicator ring will turn yellow while the chamber is warming.
   5.2.2. When the device is at temperature, the indicator ring will turn green and the display will read “Ready to Load.”
NOTE: Ensure the CellSeal vial is held only by the tubing from time of removal from LN2 until the vial is placed in the CAT. Refrain from touching the body of the vial as this can warm the outside and cause an alarm on the CAT.
5.3. Remove CellSeal vial(s) containing final product from liquid nitrogen (LN2) storage. Transport the vial on dry ice to the thaw location.
5.4. Assign expiration time/date of thawed product; 4 hours from time of thaw. 5.4.1. Record thaw time/date and expiration time/date.
5.5. Insert the CellSeal vial into the chamber, pressing down on the vial until the indicator ring turns purple and the clamping chucks close around the vial.
   5.5.1. The indicator ring will turn red if an error has occurred during the thaw process. Select Continue by tapping Enter to proceed with thaw and make note of the error in the comment box below.
5.6. As the thaw begins, the indicator will glow blue and the progress bar will be displayed.
5.7. During thaw, pass a syringe (1 mL or 3 mL), FDC, and an 18ga needle into BSC.
5.8. Attach the needle to the syringe.
5.9. Affix the remaining final product label from step 2.
5.10. When thaw is complete, the vial will be ejected from the chamber, a tone will sound, and the indicator will glow green.
   5.10.1. If an unsuccessful thaw occurred, the indicator will turn yellow and a different tone will sound. Record any error messages.
5.11. Remove the vial from the CAT and peel away the protective foil on bottom of CellSeal vial.
5.12. Swab the septum of the vial with iodine, followed by alcohol, and allow to air dry.
5.13. Cut the tubing line with the filter membrane on CellSeal vial above the filter. DO NOT cut the tubing without the filter.
5.14. Use the prepared syringe, mix well, and draw the calculated volume of cell suspension.
5.15. Remove needle and carefully cap syringe.
5.16. Remove needle and attach and FDC.
5.17. Take the prepared Normosol syringe, remove luer plug and attach to FDC on cell suspension syringe.
5.18. Mix well between syringes and push all volume to the labeled final product syringe. 5.19. Disconnect syringe containing cell suspension from FDC and attach the female end of a luer plug. Place syringe in a Ziploc/biohazard sample bag and seal.
5.20. Transfer Product:
   5.20.1. Place bag into prepared “Golden Hour” insulated container along with the Temp Logger to transport the product at ambient temperature.

Data Entry Section IV: Transfer of Products to Transport Container

| | |
|---|---|
| Time of Packaging: | Date of Packaging: |

Temp Logger Reading at Transfer (° C.):

| | |
|---|---|
| Performed By: | Verified By: |

5.20.2. Record time/date of infusion product packaging and Temp Logger Reading at the time of packaging.
   5.20.2.1. Allow probe reading to refresh for approximately 1 minute before recording the reading.
5.21. Transport product with CofA, PRF/Medication Order, and this PBR to the designated infusion location.

6. Complete transfer of custody to clinical staff
6.1. Transfer the CofA to clinical team.
6.2. Record the temp-logger min/max readings.
6.3. Open the transport container and record the date/time opened in DES V.

Data Entry Section V: Min/Max Temperature
of Container During Transport

| | |
|---|---|
| Minimum Temp (° C.): | Maximum Temp (° C.): |
| Clinical product has maintained | □ Yes |
| a temperature between | □ No, notify QA/TCPC |
| 15.5° C. to 24° C. | Leadership |
| Date/Time container opened: | |
| Performed By: | Verified By: |

(xi) Closing Paragraphs. As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventor for carrying out the invention. Of course, variations on these described embodiments will become apparent to those of ordinary skill in the art upon reading the foregoing description. The inventor expects skilled artisans to employ such variations as appropriate, and the inventor intends for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of preparing a therapeutic cell product for administration to a subject comprising
transferring a volume of therapeutic cells into a first sterile receptacle to create a cell-filled sterile receptacle;
transferring a specified volume of diluent into a second sterile receptacle to create a diluent-filled sterile receptacle;
connecting the cell-filled sterile receptacle to the diluent-filled sterile receptacle with a fluid dispensing connector; and
mixing the volume of cells with the volume of diluent thereby preparing the therapeutic cell product for administration to the subject.

2. The method of claim 1, wherein the first sterile receptacle is a syringe.

3. The method of claim 1, wherein the second sterile receptacle is a syringe.

4. The method of claim 2 or 3, wherein the syringe is fitted with a needle.

5. The method of claim 4, wherein the needle comprises a safety-shielded needle.

6. The method of claim 4, wherein the needle comprises a 14 gauge needle, 15 gauge needle, 16 gauge needle, 17 gauge needle, 18 gauge needle, 19 gauge needle, 20 gauge needle, 21 gauge needle, 22 gauge needle, 23 gauge needle, 24 gauge needle, 25 gauge needle, 26 gauge needle, or a 27 gauge needle.

7. The method of claim 4, wherein the needle comprises an 18 gauge safety-shielded needle.

8. The method of claim 4, wherein the needle is removed before connecting the cell-filled sterile receptacle to the diluent-filled sterile receptacle with a fluid dispensing connector.

9. The method of claim 1, wherein transferring the volume of cells into the first sterile receptacle comprises drawing cells into the first sterile receptacle.

10. The method of claim 1, wherein the volume of cells comprises a cell density ranging from 10×10⁶ cells/ml to 325×10⁶ cells/ml.

11. The method of claim 1, wherein transferring the specified volume of diluent into the second sterile receptacle comprises drawing diluent into the second sterile receptacle.

12. The method of claim 1, wherein a ratio of volume of cells to specified volume of diluent is selected from 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v).

13. The method of claim 1, wherein a ratio of volume of cells to specified volume of diluent is 1:10 (v/v).

14. The method of claim 13, wherein the specified volume of diluent is at least 4 times, at least 5 times, at least 6 times, at least 7 times, at least 8 times, at least 9 times, at least 10 times, at least 11 times, at least 12 times, at least 13 times, at least 14 times, or at least 15 times the volume of cells.

15. The method of claim 13, wherein the specified volume of diluent is 9 times the volume of cells.

16. The method of claim 1, wherein the diluent comprises an isotonic solution.

17. The method of claim 16, wherein the isotonic solution comprises less than 10 mg/ml sodium chloride, less than 10 mg/mL sodium acetate, less than 10 mg/mL sodium gluconate, less than 10 mg/mL potassium chloride, and less than 10 mg/mL magnesium chloride hexahydrate.

18. The method of claim 16, wherein the isotonic solution comprises 2.5-7.5 mg/mL sodium chloride, 1.5-4.0 mg/mL sodium acetate, 2.5-7.5 mg/mL sodium gluconate, 0.1-0.8 mg/mL potassium chloride, and 0.1-0.5 mg/mL magnesium chloride hexahydrate.

19. The method of claim 16, wherein the isotonic solution comprises 5.26 mg/mL sodium chloride, 2.22 mg/mL sodium acetate, 5.02 mg/mL sodium gluconate, 0.37 mg/mL potassium chloride, and 0.30 mg/mL magnesium chloride hexahydrate.

20. The method of claim 16, wherein the isotonic solution further comprises HCl for pH adjustment.

21. The method of claim 16, wherein the isotonic solution further comprises NaOH for pH adjustment.

22. The method of claim 1, wherein the fluid dispensing connector comprises at least 2 ports.

23. The method of claim 22, wherein the fluid dispensing connector comprises a first port and a second port.

24. The method of claim 23, wherein the cell-filled sterile receptacle is attached to the first port on the fluid dispensing connector and the diluent-filled sterile receptacle is attached to the second port on the fluid dispensing connector.

25. The method of claim 1, wherein mixing the volume of cells with the volume of diluent comprises forcing cells and diluent to flow between the cell-filled sterile receptacle, fluid dispensing connector, and diluent-filled sterile receptacle to create a total mixed volume of cells and diluent.

26. The method of claim 1, wherein the first sterile receptacle comprises a first syringe, the second sterile receptacle comprises a second syringe, and the mixing comprises pushing a plunger on the first syringe and the second syringe consecutively and repetitively such that the volume of cells and volume of diluent mix to create a total mixed volume of cells and diluent.

27. The method of claim 25 or 26, wherein the total mixed volume is less than 10 mL, less than 9 mL, less than 8 mL, less than 7 ml, less than 6 mL, less than 5 mL, less than 4 mL, less than 3 mL, less than 2 mL, or less than 1 mL.

28. The method of claim 27, wherein the total mixed volume is less than 5 mL.

29. The method of claim 25, comprising transferring the total mixed volume into either the cell-filled sterile receptacle or the diluent-filled sterile receptacle to create a product-filled sterile receptacle.

30. The method of claim 29, comprising transferring the total mixed volume into the cell-filled sterile receptacle to create the product-filled sterile receptacle.

31. The method of claim 29, comprising transferring the total mixed volume into the diluent-filled sterile receptacle to create the product-filled sterile receptacle.

32. The method of claim 29, comprising removing the product-filled sterile receptacle from the fluid dispensing connector.

33. The method of claim 29, comprising plugging the product-filled sterile receptacle with luer plug.

34. The method of claim 29, comprising storing the product-filled sterile receptacle in a vessel on dry ice.

35. The method of claim 29, comprising transferring the product-filled sterile receptacle to a clinic for administration to the subject.

36. The method of claim 1, wherein the subject has cancer.

37. The method of claim 36, wherein the cancer is a central nervous system tumor, glioma, ependymoma, medulloblastoma, germ cell tumor, atypical teratoid, primitive neuroectodermal tumor, choroid plexus carcinoma, or pineoblastoma.

38. The method of claim 36, wherein the cancer is a central nervous system tumor.

39. The method of claim 1, wherein the therapeutic cells comprise cells from the subject.

40. The method of claim 39, wherein the cells from the subject comprise immune cells.

41. The method of claim 40, wherein the immune cells comprise T cells.

42. The method of claim 41, wherein the T cells are CD4+ T cells.

43. The method of claim 41, wherein the T cells are CD8+ T cells.

44. The method of claim 41, wherein the T cells are CD4+ and CD8+ T cells.

45. The method of claim 41, wherein the T cells are genetically modified to express a therapeutic molecule.

46. The method of claim 45, wherein the therapeutic molecule comprises a chimeric antigen receptor (CAR).

47. The method of claim 41, wherein the T cells are genetically modified to express a CAR and EGFRt.

48. The method of claim 46 or 47, wherein the CAR is an anti-Her2 CAR.

49. The method of claim 1, further comprising administering the therapeutic cell product to the subject.

50. The method of claim 49, wherein the administering comprises injection, infusion, implantation, or transplantation.

51. The method of claim 49, wherein the administering comprises injecting the therapeutic cell product at the site of a tumor resection in the subject.

52. The method of claim 49, wherein the administering comprises injecting the therapeutic cell product into the ventricular system of the central nervous system of the subject.

53. The method of claim 49, wherein the administering comprises infusion.

54. The method of claim 1, wherein the subject is greater than 1 year old, greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, greater than 16 years old, greater than 17 years old, greater than 18 years old, greater than 19 years old, greater than 20 years old, greater than 21 years old, greater than 22 years old, greater than 23 years old, greater than 24 years old, or greater than 25 years old.

55. The method of claim 1, wherein the subject is greater than 1 year old.

56. The method of claim 1, wherein the subject is less than 26 years old.

57. The method of claim 1, wherein the subject is greater than 26 years old.

58. The method of claim 1, wherein the method does not comprise centrifugation.

59. The method of claim 1, wherein the method does not comprise sterile welding.

60. The method of claim 1, wherein the method does not comprise a wash step.

61. The method of claim 1, wherein the method further comprises cryopreserving the therapeutic cells to create cryopreserved cells and thawing the cryopreserved cells to create thawed therapeutic cells before transferring the volume of thawed therapeutic cells into the first sterile receptacle.

62. The method of claim 61, wherein the cryopreserving comprises suspending the therapeutic cells in a freezing medium and reducing the temperature to a cryopreservation temperature to create cryopreserved cells.

63. The method of claim 62, wherein the freezing medium comprises a cryopreservative.

64. The method of claim 63, wherein the cryopreservative is DMSO.

65. The method of claim 62, wherein the freezing medium comprises 5% DMSO.

66. The method of claim 62, wherein the cryopreservation temperature ranges from −205° C. to −60° C.

67. The method of claim 62, wherein the cryopreserved cells are stored in liquid nitrogen.

68. The method of claim 61, wherein the thawing comprises incubating the cryopreserved cells at progressively increasing temperatures.

69. The method of claim 68, wherein the incubating comprises putting the cryopreserved cells in a temperature controlled incubator.

70. The method of claim 69, wherein the temperature controlled incubator comprises a controlled water bath, a temperature controlled oven, or an automated thawing device.

71. The method of claim 61, wherein the thawing comprises using an automated thawing device.

72. The method of claim 61, wherein the thawing comprises incubating the cryopreserved cells at a temperature ranging from 15° C. to 30° C.

73. The method of claim 61, wherein the thawing comprises incubating the cryopreserved cells at a temperature ranging from 30° C. to 45° C.

74. The method of claim 61, wherein the thawing comprises incubating the cryopreserved cells at a temperature of 37° C.

75. The method of claim 61, further comprising determining a viability of the thawed therapeutic cells.

76. The method of claim 75, wherein the viability of thawed therapeutic cells is greater than 65%.

77. The method of claim 75, wherein the viability of thawed therapeutic cells is greater than 70%.

78. A method for formulation of therapeutic cells for administration to a subject comprising thawing cryopreserved cells to create thawed cells; transferring a volume of thawed cells into a first syringe to create a cell-filled syringe; transferring a specified volume of diluent into a second syringe to create a diluent-filled syringe; connecting the cell-filled syringe to the diluent-filled syringe with a fluid dispensing connector; mixing the volume of cells with the volume of diluent to create a total mixed volume; and transferring the total mixed volume into either the cell-filled syringe or the diluent-filled syringe.

79. The method of claim 78, wherein the thawing comprises using an automated thawing device.

80. The method of claim 78, comprising a ratio of volume of thawed cells to volume of diluent of 1:5 (v/v), 1:6 (v/v), 1:7 (v/v), 1:8 (v/v), 1:9 (v/v), 1:10 (v/v), 1:11 (v/v), 1:12 (v/v), 1:13 (v/v), 1:14 (v/v), 1:15 (v/v), or 1:16 (v/v).

81. The method of claim 78, comprising a ratio of volume of thawed cells to volume of diluent of 1:10 (v/v).

82. The method of claim 78, wherein the total mixed volume is transferred to the cell-filled syringe.

83. The method of claim 78, comprising disconnecting the fluid dispensing connector from the cell-filled syringe and plugging the cell-filled syringe with a luer plug.

84. The method of claim 78, wherein the total mixed volume is transferred to the diluent-filled syringe.

85. The method of claim 78, comprising disconnecting the fluid dispensing connector from the diluent-filled syringe and plugging the diluent-filled syringe with a luer plug.

86. A kit for dilution of cell product comprising syringes, needles, fluid dispensing connector, and diluent.

87. The kit of claim 86, comprising at least two syringes.

88. The kit of claim 86, wherein the needles attach to the syringes.

89. The kit of claim 86, wherein the needles comprise 18 gauge safety-shielded needles.

90. The kit of claim 86, wherein the fluid dispensing connector attaches to the syringes.

91. The kit of claim 86, wherein the diluent comprises an isotonic solution.

92. The kit of claim 91, wherein the isotonic solution comprises 5.26 mg/mL sodium chloride, 2.22 mg/mL sodium acetate, 5.02 mg/mL sodium gluconate, 0.37 mg/mL potassium chloride, and 0.30 mg/mL magnesium chloride hexahydrate.

93. The kit of claim 86, comprising NaOH and/or HCl for pH adjustment.

94. The kit of claim 86, comprising a luer plug.

95. The kit of claim 86, comprising disinfectant.

96. The kit of claim 95, wherein the disinfectant comprises povidone-iodine, isopropyl alcohol, ethanol, methanol, and/or hydrogen peroxide.

97. The kit of claim 95, wherein the disinfectant comprises povidone-iodine and/or isopropyl alcohol swabs.

98. The kit of claim 86, comprising instructions for dilution of cell product.
